(19) United States
(12) Patent Application Publication
(10) Pub. No.: US 2022/0018741 A1
(43) Pub. Date: Jan. 20, 2022

(54) HIGH/HYPERVELOCITY PARTICLE CAPTURE AND ANALYSIS METHOD AND APPARATUS

(71) Applicant: The Regents of the University of California, Oakland, CA (US)

(72) Inventors: Richard A. Mathies, Walnut Creek, CA (US); Anna Butterworth, Berkeley, CA (US); Matin Golozar, Berkeley, CA (US); Jeremy McCauley, Martinez, CA (US); James Stephen Oliver New, London (GB)

(73) Assignee: The Regents of the University of California, Oakland, CA (US)

(21) Appl. No.: 17/298,502

(22) PCT Filed: Dec. 2, 2019
(86) PCT No.: PCT/US2019/064057

§ 371 Date: May 28, 2021

Related U.S. Application Data
(60) Provisional application No. 62/774,786, filed on Dec. 3, 2018.

(51) Int. Cl. G01N 1/22; G01N 1/40; B01L 1/00; G01N 35/10; B01L 3/00; G01N 33/68; B64D 47/00; B64G 1/66

(52) U.S. Cl. CPC G01N 1/2273; B01L 2300/0883; B01L 1/00; G01N 35/1095; B01L 3/502715; B01L 3/567; B01L 3/52; G01N 33/6809; B64D 47/00; B64G 1/66; G01N 2001/2279; G01N 2001/4061; B01L 2300/043; B01L 2300/045; B01L 2400/0487; B01L 2400/06; B01L 2400/0421; B01L 2200/16; B01L 2300/088; G01N 1/4055

(57) ABSTRACT

In various embodiments a capture surface for capturing high velocity and hypervelocity dust and ice particles is provided. In certain embodiments the capture surface is comprised of a soft metal that is chosen to optimize particle capture efficiency, to minimize thermal degradation of chemicals and biochemical in the particles, and to present the captured particles to an analyzer for chemical and biochemical analysis of the particles and their contents. In various embodiments capture chambers comprising one or more such capture surfaces are provided as well as methods of use thereof.

### CROSS-REFERENCE TO RELATED APPLICATIONS

This application claims benefit of and priority to U.S. Ser. No. 62/774,786, filed on Dec. 3, 2018, which is incorporated herein by reference in its entirety for all purposes.

### STATEMENT OF GOVERNMENTAL SUPPORT

This invention was made with government support under Grant No. 80NSSC17K0600 awarded by the National Aeronautics and Space Administration. The Government has certain rights in this invention.

### BACKGROUND

It has long been desired to analyze extraterrestrial materials for organic molecules that can be indicative of habitability as well as past or present life. Such extraterrestrial materials, include for example, extraterrestrial cloud, dust, aerosol, and plume samples.

One previous approach for analyzing such samples was Stardust, which was a passive return mission that captured cometary dust for later laboratory analysis on Earth. The organic analyses on Stardust have included synchrotron X-ray spectroscopy on particles extracted from aerogel (Sandford et al. (2006) Science, 314: 1720-1724; De Gregorio et al. (2011) Meteroitics and Planetary Sci. 46: 1376-1396), dissolution of foil residue for organic analyses (Elsila et al. (2009) Meteoritics and Planetary Sci. 44: 1323-1330; Glavin et al. (2008) Meteoritics and Planetary Sci. 42: 399-413), and non-destructive microprobe analyses (Wozniakiewiz et al. (2018) Meteoritics and Planetary Sci. 52: 1066-1080). All of these techniques require sample return to large laboratory-based facilities on Earth, rather than in situ integrated collection and analysis. Also, detectors capable of chemical analyses have been applied to returned surfaces from the Long Duration Exposure Facility (LDEF), the Space Flyer Unit and the Hubble Space Telescope (Graham, et al. (2001) In Space Debris, 473: 197-202).

The Cosmic Dust Analyzer (CDA) is a good example of a fully in situ chemical analyzer. The CDA, however, used a mass spectrometer to analyze plasma, containing ions and electrons, released during destructive intense impacts between the particles and target material (Bradley et al. (1996) SPIE Proc. 2803: 108-118) and was not suitable for in situ organic detection.

### SUMMARY

In various embodiments a capture and analysis system is provided that efficiently captures high velocity particles (e.g., high velocity plume ice particles), does not degrade the entrained organic molecules, that can be effectively and efficiently analyzed, that can be readily cleaned to provide low background and forward contamination, and that has high sensitivity for analyzing the trace organics.

Various embodiments contemplated herein may include, but need not be limited to, one or more of the following:

Embodiment 1: A particle capture surface configured for capture of high and/or hyper velocity dust, aerosol, and/or ice particles, wherein said capture surface is comprised of soft metal that maximizes particle capture efficiency, minimizes thermal degradation and shock degradation of chemical and biochemical components in the particles, and said surface is configured to present the captured particles, or components therein, on said surface for direct analysis or to deliver said particles, or component therein, to an analyzer for chemical and/or biochemical analysis of the particles and their component contents.

Embodiment 2: The particle capture surface of embodiment 1, wherein said capture surface is comprised of a soft metal that maximizes particle capture efficiency, minimizes thermal degradation and shock degradation of chemicals and biochemicals in the particles, and said surface is configured to present the captured particles on said surface for direct analysis or to deliver said particles, or component thereof, to an analyzer for chemical and/or biochemical analysis of the particles and their contents.

Embodiment 3: The particle capture surface according to any one of embodiments 1-2, wherein said surface is configured to deliver said particles to an analyzer for chemical and/or biochemical analysis of the particles and their contents.

Embodiment 4: The particle capture surface according to any one of embodiments 1-3, wherein said surface is configured to capture extraterrestrial dust, aerosol, and/or ice particles.

Embodiment 5: The particle capture surface according to any one of embodiments 1-3, wherein said surface is configured to capture extraterrestrial dust, aerosol, and/or ice particles in high earth orbit.

Embodiment 6: The particle capture surface according to any one of embodiments 1-3, wherein said surface is configured to capture extraterrestrial dust, aerosol, and/or ice particles at high altitude.

Embodiment 7: The particle capture surface according to any one of embodiments 1-6, wherein said capture surface is configured to provide a particle capture efficiency of at least 0.01%, or at least 0.1%, or at least 0.5%, or at least 1%, or at least 10%, or at least 30%, or at least 50%, or at least 80%, or at least 90% for particles, or at least 95%, or at least 98% up to 100%.

Embodiment 8: The particle capture surface of embodiment 7, wherein said capture surface is configured to provide a particle capture efficiency ranging from about 1% up to about 50%.

Embodiment 9: The particle capture surface according to any one of embodiments 7-8, wherein said capture efficiency is for particles impacting said capture surface at an angle ranging from about 45 degrees to about 90 degrees.

Embodiment 10: The particle capture surface of embodiment 9, wherein said capture efficiency is for particles impacting said capture surface at an angle of about 90 degrees.

Embodiment 11: The particle capture surface according to any one of embodiments 1-10, wherein said surface is configured to perform said capturing at an average relative velocity of said capture surface and dust and ice particles ranging from about 1 m/s, or from about 10 m/s, or from about 100 m/s, or from about 500 m/s, or from about 1 km/s, up to about 10 km/s, or up to about 5 km/s, or up to about 2.5 km/s, or up to about 1 km/s.

Embodiment 12: The particle capture surface of embodiment 11, wherein said surface is configured to perform said capturing at an average relative velocity of said capture surface and dust and ice particles ranging from about 1 m/s up to about 5 km/s, or from about 100 m/s up to about 5 km/s, or from about 500 m/s up to about 1 km/s up to about 5 km/s.

Embodiment 13: The particle capture surface according to any one of embodiments 1-12, wherein said thermal degradation and shock degradation is sufficiently low to permit dispositive identification of at least about 5%, or at least about 10%, or at least about 20%, or at least about 30%, or at least about 40%, or at least about 50%, or at least about 60%, or at least about 70%, or at least about 80%, or at least about 90%, or at least about 95%, or at least about 98% of the organic compounds captured on said surface.

Embodiment 14: The particle capture surface of embodiment 13, wherein said dispositive identification is by Raman spectroscopy.

Embodiment 15: The particle capture surface of embodiment 13, wherein said dispositive identification is by optical absorption or emission microscopy or SEM.

Embodiment 16: The particle capture surface of embodiment 13, wherein said dispositive identification is by a programmable microfluidic analyzer (PMA).

Embodiment 17: The particle capture surface of embodiment 13, wherein said dispositive identification is by a mass spectroscopy (e.g., laser desorption mass spectroscopy).

Embodiment 18: The particle capture surface according to any one of embodiments 1-17, wherein said surface is configured to capture particles impacting said surface an angle between about 45 degrees and about 90 degrees.

Embodiment 19: The particle capture surface according to any one of embodiments 1-18, wherein the average size of said aerosol, ice or dust particles ranges from about 0.1 μm, or from about 1 μm, or from about 2 μm up to about 1000 μm, or up to about 500 μm, or up to about 100 μm, or up to about 50 μm, or up to about 20 μm in diameter.

Embodiment 20: The particle capture surface of embodiment 19, wherein the average size of said aerosol, ice or dust particles ranges from about 0.1 μm up to about 20 μm.

Embodiment 21: The particle capture surface according to any one of embodiments 1-20, wherein the projected area of said capture surface area ranges from about 1 cm², or from about 5 cm², or from about 10 cm², or from about 20 cm², or from about 30 cm², or from about 40 cm², or about 50 cm², or from about 60 cm², or from about 70 cm², or from about 80 cm², or from about 90 cm², or from about 100 cm², up to about 1,000 cm², or up to about 500 cm², or up to about 400 cm², or up to about 300 cm², or up to about 200 cm², or up about 190 cm², or up to about 180 cm², or up to about 170 cm², or up to about 160 cm², or up to about 150 cm².

Embodiment 22: The particle capture surface of embodiment 21, wherein the projected area of said capture surface ranges from about 10 cm² up to about 200 cm², or from about 20 cm² up to about 150 cm², or from about 50 cm² up to about 120 cm².

Embodiment 23: The particle capture surface according to any one of embodiments 1-22, wherein the shape of the projected area of said capture surface comprise a shape selected from the group consisting of circular, triangular, square, rectangular, hexagonal, and the like.

Embodiment 24: The particle capture surface of embodiment 23, wherein the shape of the projected area of said capture surface is circular.

Embodiment 25: The particle capture surface of embodiment 24, wherein the projected area of said capture surface has a diameter of about 10 cm.

Embodiment 26: The particle capture surface according to any one of embodiments 1-25, wherein said soft capture surface is comprised of a metal selected from the group consisting of Al, Au, Ag, Cu, mercury, gallium, indium, lead, brass, and bronze, or any other soft metal or alloy with similar mechanical properties.

Embodiment 27: The particle capture surface according to any one of embodiments 1-26, wherein said capture surface is comprised of one, or two or more different soft metal layers where the metals and their thicknesses simultaneously provide both efficient capture and minimal degradation of the chemicals in the particles.

Embodiment 28: The particle capture surface of embodiment 27, wherein one or more of said layers ranges in thickness from about a few microns up to about a few mm.

Embodiment 29: The particle capture surface of embodiment 27, wherein one or more of said layers ranges in thickness from about 1 μm, or from about 2 μm, or from about 5 μm, or from about 10 μm, or from about 20 μm, or from about 50 μm, or from about 100 μm, or from about 500 μm up to about 10 mm, or up to about 5 mm, or up to about 4 mm, or up to about 3 mm, or up to about 2 mm, or up to about 1 mm.

Embodiment 30: The particle capture surface according to any one of embodiments 1-29, wherein said particle capture surface comprises a soft metal disposed on top of a harder metal or a silica substrate.

Embodiment 31: The particle capture surface of embodiment 30, wherein said particle capture surface comprises a soft metal disposed on top of a harder metal or other material.

Embodiment 32: The particle capture surface of embodiment 31, wherein said particle capture surface comprises a gold layer disposed on an aluminum and/or silver layer.

Embodiment 33: The particle capture surface of embodiment 32, wherein said particle capture surface comprises a gold layer disposed on an aluminum layer.

Embodiment 34: The particle capture surface according to any one of embodiments 1-33 wherein said capture surface is configured to present captured particles for chemical and biochemical assay by optical spectroscopy, optical microscopy, SEM, or mass spectrometry.

Embodiment 35: The particle capture surface according to any one of embodiments 1-34, wherein said capture surface is configured to present captured particles for chemical and biochemical assay by Raman spectroscopy or Raman microscopy.

Embodiment 36: The particle capture surface according to any one of embodiments 1-35, wherein said capture surface comprises 2 or more, or 3 or more, or 4 or more or 5 or more different regions comprising different materials and/or material thicknesses to produce different hardnesses.

Embodiment 37: The particle capture surface of embodiment 36, wherein said capture surface comprises 2 or more, or 3 or more, or 4 or more or 5 or more different regions comprising different materials and/or material thicknesses to simultaneously provide optimal capture of particles having different velocities.

Embodiment 38: The particle capture surface according to any one of embodiments 1-35, wherein metals comprising said capture surface vary in thickness and/or composition to provide a gradient in hardness across said surface.

Embodiment 39: The particle capture surface according to any one of embodiments 1-38, wherein said capture surface comprises a component in an aircraft, rocket, satellite or space probe.

Embodiment 40: A particle capture surface configured for capture of high and/or hyper velocity dust, aerosol, and/or ice particles, wherein said capture surface is comprised of an easily cleaned soft metal that maximizes particle capture efficiency, minimizes thermal degradation of chemicals and biochemicals in the particles, that is configured to permit facile dissolution of the particles and their chemical and biochemical contents into a volume of extractant fluid, and is configured to enable transfer of the extractant fluid to an analyzer for chemical and biochemical analysis.

Embodiment 41: The particle capture surface of embodiment 40, wherein said surface is configured to capture extraterrestrial dust, aerosol, and/or ice particles.

Embodiment 42: The particle capture surface of embodiment 40, wherein said surface is configured to capture extraterrestrial dust, aerosol, and/or ice particles in high earth orbit.

Embodiment 43: The particle capture surface of embodiment 40, wherein said surface is configured to capture extraterrestrial dust, aerosol, and/or ice particles at high altitude.

Embodiment 44: The particle capture surface according to any one of embodiments 40-43, wherein said capture surface is configured to provide a surface in an open chamber configured to pass fluid across said surface to surface to dissolve chemical/biochemical contents of said particles.

Embodiment 45: The particle capture surface according to any one of embodiments 40-43, wherein said capture surface comprises features that, when said surface is capped with a lid, said features provide one or more channels that direct the flow of a fluid over said surface to dissolve chemical/biochemical contents of said particles.

Embodiment 46: The particle capture surface of embodiment 45, wherein said one or more channels comprise a serpentine channel that directs flow from an inlet port to an outlet port.

Embodiment 47: The particle capture surface of embodiment 46, wherein said one or more channels comprise a spiral channel pattern that directs flow from an inlet port to an outlet port.

Embodiment 48: The particle capture surface of embodiment 47, wherein said one or more channels comprise a square spiral serpentine channel.

Embodiment 49: The particle capture surface of embodiment 47, wherein said one or more channels comprise a circular spiral serpentine channel.

Embodiment 50: The particle capture surface of embodiment 47, wherein said one or more channels comprise a switchback serpentine channel.

Embodiment 51: The particle capture surface of embodiment 45, wherein said one or more channels comprise a branched channel pattern that directs flow from an inlet port to an outlet port.

Embodiment 52: The particle capture surface according to any of embodiments 45-51, wherein said one or more channels range in depth from about 10 μm, or from about 20 μm, or from about 30 μm, or from about 40 μm up to about 300 μm, or up to about 200 μm, or up to about 100 μm, or up to about 70 μm, or up to about 60 μm, or up to about 50 μm, or up to about 30 μm.

Embodiment 53: The particle capture surface according to any of embodiments 45-52, wherein said one or more channels range in width from about 20 μm, or from about 30 μm, or from about 40 μm, or from about 50 μm up to about 1000 μm, or to about 500 μm, or up to about 200 μm, or up to about 100 μm.

Embodiment 54: The particle capture surface according to any of embodiments 45-52, wherein said one or more channels have a depth of about 100 μm and a width of about 400 μm.

Embodiment 55: The particle capture surface according to any one of embodiments 53-54, wherein said channels have a spacing (between channels) of about 125 μm.

Embodiment 56: The particle capture surface according to any of embodiments 45-55, wherein said one or more channels have a square or rectangular cross-section, a cross-section with chamfered sides, a cross-section with a curved bottom, and a cross-section with sloping sides, or a conical cross-section.

Embodiment 57: The particle capture surface of embodiment 56, wherein said one or more channels have a cross-section that is not square or rectangular.

Embodiment 58: The particle capture surface of embodiment 57, wherein said one or more channels have a cross-section with chamfered sides, a cross-section with a curved bottom, and a cross-section with sloping sides, or a conical cross-section.

Embodiment 59: The particle capture surface according to any of embodiments 45-58, wherein said features comprise a compliant top coat to improve sealing to a juxtaposed surface.

Embodiment 60: The particle capture surface of embodiment 59, wherein said compliant top coat comprises a gasket material.

Embodiment 61: The particle capture surface of embodiment 60, wherein said compliant top coat comprises a soft metal gasket material.

Embodiment 62: The particle capture surface of embodiment 59, wherein said soft metal gasket material comprises indium.

Embodiment 63: The particle capture surface according to any of embodiments 45-62, wherein, wherein said features comprise a hydrophobic barrier that prevents wetting in a thin gap between the features and a juxtaposed surface.

Embodiment 64: The particle capture surface of embodiment 62, wherein said hydrophobic barrier is comprised of a gold overcoat with a hydrophobic thiol coating.

Embodiment 65: The particle capture surface according to any one of embodiments 40-64, wherein said capture surface is configured to provide a particle capture efficiency of at least 0.01%, or at least 0.1%, or at least 0.5%, or at least 1% cm², or at least 10% cm², or at least 30% cm², or at least 50% cm², or at least 80% cm², of at least 90% for particles cm², or at least 95% cm², or at least 98%.

Embodiment 66: The particle capture surface of embodiment 65, wherein said capture surface is configured to provide a particle capture efficiency ranging from about 1% up to about 50%.

Embodiment 67: The particle capture surface according to any one of embodiments 65-66, wherein said capture efficiency is for particles impacting said capture surface at an angle ranging from about 45 degrees to about 90 degrees.

Embodiment 68: The particle capture surface of embodiment 67, wherein said capture efficiency is for particles impacting said capture surface at an angle of about 90 degrees.

Embodiment 69: The particle capture surface according to any one of embodiments 65-68, wherein said surface is configured to perform said capturing at an average relative velocity of said capture surface and dust and ice particles ranging from about 1 m/s, or from about 10 m/s, or from about 100 m/s, or from about 500 m/s, or from about 1 km/s, up to about 10 km/s, or up to about 5 km/s, or up to about 2.5 km/s, or up to about 1 km/s.

Embodiment 70: The particle capture surface of embodiment 69, wherein said surface is configured to perform said capturing at an average relative velocity of said capture surface and dust and ice particles ranging from about 1 m/s up to about 5 km/s, or from about 100 m/s up to about 5 km/s, or from about 500 m/s up to about 1 km/s up to about 5 km/s.

Embodiment 71: The particle capture surface according to any one of embodiments 65-70, wherein said thermal degradation is sufficiently low to permit dispositive identification of at least about 5%, or at least about 10%, or at least about 20%, or at least about 30%, or at least about 40%, or at least about 50%, or at least about 60%, or at least about 70%, or at least about 80%, or at least about 90%, or at least about 95%, or at least about 98% of the organic compounds captured on said surface.

Embodiment 72: The particle capture surface of embodiment 71, wherein said dispositive identification is by Raman spectroscopy.

Embodiment 73: The particle capture surface of embodiment 71, wherein said dispositive identification is by optical microscopy or SEM.

Embodiment 74: The particle capture surface of embodiment 71, wherein said dispositive identification is by mass spectroscopy (e.g., laser desorption mass spectroscopy).

Embodiment 75: The particle capture surface of embodiment 71, wherein said dispositive identification is by a programmable microfluidic analyzer (PMA).

Embodiment 76: The particle capture surface of embodiment 71, wherein said dispositive identification is by a mass spectroscopy (e.g., laser desorption mass spectroscopy).

Embodiment 77: The particle capture surface according to any one of embodiments 65-76, wherein the average size of said aerosol, ice or dust particles ranges from about 0.1 μm, or from about 1 μm, or from about 2 μm up to about 1000 μm, or up to about 500 μm, or up to about 100 μm, or up to about 50 μm, or up to about 20 μm in diameter.

Embodiment 78: The particle capture surface of embodiment 77, wherein the average size of said aerosol, ice or dust particles ranges from about 0.1 μm up to about 20 μm.

Embodiment 79: The particle capture surface according to any one of embodiments 40-78, wherein said surface is configured to capture particles impacting said surface an angle between about 45 degrees and about 90 degrees.

Embodiment 80: The particle capture surface according to any one of embodiments 65-79, wherein the projected area of said capture surface area ranges from about 1 cm², or from about 5 cm², or from about 10 cm², or from about 20 cm², or from about 30 cm², or from about 40 cm², or about 50 cm², or from about 60 cm², or from about 70 cm², or from about 80 cm², or from about 90 cm², or from about 100 cm², up to about 1000 cm², or up to about 500 cm², or up to about 400 cm², or up to about 300 cm², or up to about 200 cm², or up about 190 cm², or up to about 180 cm², or up to about 170 cm², or up to about 160 cm², or up to about 150 cm².

Embodiment 81: The particle capture surface of embodiment 80, wherein the projected area of said capture surface ranges from about 10 cm² up to about 200 cm², or from about 20 cm² up to about 150 cm², or from about 50 cm² up to about 120 cm².

Embodiment 82: The particle capture surface according to any one of embodiments 40-81, wherein the shape of the projected area of said capture surface comprises a shape selected from the group consisting of circular, triangular, square, rectangular, and hexagonal.

Embodiment 83: The particle capture surface of embodiment 82, wherein the shape of the projected area of said capture surface is circular.

Embodiment 84: The particle capture surface of embodiment 83, wherein the projected area of said capture surface has a diameter of about 10 cm.

Embodiment 85: The particle capture surface according to any one of embodiments 40-84, wherein said soft capture surface is comprised of a metal selected from the group consisting of Al, Au, Ag, Cu, mercury, gallium, indium, lead, brass, and bronze, or any other soft metal or alloy with similar mechanical properties.

Embodiment 86: The particle capture surface according to any one of embodiments 40-85, wherein said capture surface is comprised of one, or two or more different soft metal layers where the metals and their thicknesses simultaneously provide both efficient capture and minimal degradation of the chemicals in the particles.

Embodiment 87: The particle capture surface of embodiment 86, wherein one or more of said layers ranges in thickness from about a few microns up to about a few mm.

Embodiment 88: The particle capture surface of embodiment 86, wherein one or more of said layers ranges in thickness from about 1 μm, or from about 2 μm, or from about 5 μm, or from about 10 μm, or from about 20 μm, or from about 50 μm, or from about 100 μm, or from about 500 μm up to about 10 mm, or up to about 5 mm, or up to about 4 mm, or up to about 3 mm, or up to about 2 mm, or up to about 1 mm.

Embodiment 89: The particle capture surface according to any one of embodiments 40-88, wherein said particle capture surface comprises a soft metal disposed on top of a harder metal or a silica substrate.

Embodiment 90: The particle capture surface of embodiment 89, wherein said particle capture surface comprises a soft metal disposed on top of a harder metal.

Embodiment 91: The particle capture surface of embodiment 90, wherein said particle capture surface comprises a gold layer disposed on an aluminum and/or silver layer.

Embodiment 92: The particle capture surface of embodiment 91, wherein said particle capture surface comprises a gold layer disposed on an aluminum layer.

Embodiment 93: The particle capture surface of embodiment 89, wherein said particle capture surface comprise a gold layer disposed on an aluminum and/or silver layer.

Embodiment 94: The particle capture surface according to any one of embodiments 40-93, wherein said capture surface comprises 2 or more, or 3 or more, or 4 or more or 5 or more different regions comprising different materials and/or material thicknesses to produce different hardnesses.

Embodiment 95: The particle capture surface of embodiment 94, wherein said capture surface comprises 2 or more, or 3 or more, or 4 or more or 5 or more different regions comprising different materials and/or material thicknesses to simultaneously provide optimal capture of particles having different velocities.

Embodiment 96: The particle capture surface according to any one of embodiments 40-93, wherein metals comprising said capture surface various in thickness and/or composition to provide a gradient in hardness across said surface.

Embodiment 97: The particle capture surface according to any one of embodiments 40-96, wherein said capture surface comprises a component in an aircraft, rocket, satellite or space probe.

Embodiment 98: A particle capture chamber for capture of high velocity dust and ice particles, said chamber comprising: a first particle capture surface according to any one of embodiments 1-38; and a moveable lid where said lid is configured so that when said capture chamber is closed said lid covers said particle capture surface and with said capture surface forms a sample chamber.

Embodiment 99: The particle capture chamber of embodiment 98, wherein said surface is configured to capture extraterrestrial dust, aerosol, and/or ice particles.

Embodiment 100: The particle capture chamber of embodiment 98, wherein said surface is configured to capture extraterrestrial dust, aerosol, and/or ice particles in high earth orbit.

Embodiment 101: The particle capture chamber of embodiment 98, wherein said surface is configured to capture extraterrestrial dust, aerosol, and/or ice particles at high altitude.

Embodiment 102: The particle capture chamber according to any one of embodiments 98-101, wherein said lid is configured to slide open.

Embodiment 103: The particle capture chamber according to any one of embodiments 98-101, wherein said lid is hinged such that it can open providing enhanced material capture by permitting particle capture on said first particle capture surface and on a second particle capture surface disposed on said lid, wherein said second particle capture surface also comprises a particle capture surface according to any one of embodiments 1-38.

Embodiment 104: The particle capture chamber of embodiment 103, wherein said first particle capture surface and said second particle capture surface are the same materials and configuration.

Embodiment 105: The particle capture chamber of embodiment 103, wherein said first particle capture surface and said second particle capture surface are the different materials and/or configuration.

Embodiment 106: The particle capture chamber according to any one of embodiments 98-105, wherein said sample chamber is configured with an inlet and outlet port and is configured to wash said first capture surface and, when present said second capture surface, and deliver dust and ice particles and their contents to a programmable microfluidic analyzer (PMA) operably coupled to said capture chamber.

Embodiment 107: The particle capture chamber of embodiment 106, wherein said PMA comprises: a plurality of pneumatic inputs; a plurality of microfluidic channels; and a plurality of μCE separation channels, where said pneumatic inputs microfluidic channels and μCE separation channels are configured to so that fluid samples enter and leave the processor through access ports; wherein an array of valves drive fluid routing on the PMA; and sample and reagent storage is provided in addressable wells at the top.

Embodiment 108: The particle capture chamber of embodiment 107, wherein said PMA permits analysis of different samples.

Embodiment 109: The particle capture chamber according to any one of embodiments 98-108, wherein said capture surface comprises a component in an aircraft, a rocket, a satellite or space probe.

Embodiment 110: A particle capture chamber for capture of high velocity dust and ice particles, said chamber comprising: a first particle capture surface according to any one of embodiments 40-93; and a moveable lid where said lid is configured so that when said capture chamber is closed said lid covers said particle capture surface and with said capture surface forms a sample chamber that permits facile dissolution of the particles and their chemical and biochemical contents into a volume of extractant fluid and that enables transfer of the extractant fluid to an analyzer for chemical/biochemical analysis.

Embodiment 111: The particle capture chamber of embodiment 110, wherein said surface is configured to capture extraterrestrial dust, aerosol, and/or ice particles.

Embodiment 112: The particle capture chamber of embodiment 110, wherein said surface is configured to capture extraterrestrial dust, aerosol, and/or ice particles in high earth orbit.

Embodiment 113: The particle capture chamber of embodiment 110, wherein said surface is configured to capture extraterrestrial dust, aerosol, and/or ice particles at high altitude.

Embodiment 114: The particle capture chamber according to any one of embodiments 110-113, wherein said lid is configured to slide open.

Embodiment 115: The particle capture chamber according to any one of embodiments 110-113, wherein said lid is hinged such that it can open providing enhanced material capture by permitting particle capture on said first particle capture surface and on a second particle capture surface disposed on said lid, wherein said second particle capture surface comprises a particle capture surface according to any one of embodiments 1-38 or a particle capture surface according to any one of embodiments 40-93.

Embodiment 116: The particle capture chamber of embodiment 115, wherein said first particle capture surface and said second particle capture surface are the same materials and configuration.

Embodiment 117: The particle capture chamber of embodiment 115, wherein said first particle capture surface and said second particle capture surface are the different materials and/or configuration.

Embodiment 118: The particle capture chamber according to any one of embodiments 115-117, wherein said lid is configured so that when closed, microchannels in said first particle capture surface are sealed, and when present in said second particle capture surface microchannels in said second particle capture surface are sealed.

Embodiment 119: The particle capture chamber according to any one of embodiments 115-118, wherein said sample chamber is configured to direct the flow of extractant fluid through the chamber so that the chemical/biochemical contents are dissolved in an extractant fluid volume smaller than the total volume of the chamber of said chamber without the microchannels present thereby concentrating said chemical/biochemical contents.

Embodiment 120: The particle capture chamber of embodiment 119, wherein the extractant fluid volume is less than 10%, or less than about 5%, or less than about 2% of the total volume of the chamber without the microchannels present.

Embodiment 121: The particle capture chamber according to any one of embodiments 119-120, wherein the concentration of analyte in said extractant fluid is increased by at least at least 2-fold, or at least about 5-fold, or at least about 10-fold, or at least about 20-fold as compared to the concentration of said analyte present in a volume of extractant fluid equal to the total volume of said chamber.

Embodiment 122: The particle capture chamber according to any one of embodiments 119-120, wherein the wherein the increase in concentration of the analyte provides for a 10-fold, or at least about a 20-fold improvement so that the extractant volume is 1/10 or 1/20 or less of the nominal volume of the chamber without the channels.

Embodiment 123: The particle capture chamber according to any one of embodiments of embodiment 119-122, wherein said first capture surface and, when present, said second capture surface, comprises channels that can be effectively washed with a total volume of extractant fluid of less than about 100 μL, or less than about 75 μL, or less than about 50 μL, or less than about 40 μL, or less than about 30 μL, or less than about 20 μL, or less than about 15 μL.

Embodiment 124: The particle capture chamber of embodiment 123, wherein said first capture surface and, when present, said second capture surface, comprises channels that can be effectively washed with a total volume of extractant fluid of as low as 10 μL or less.

Embodiment 125: The particle capture chamber according to any one of embodiments 115-124, wherein said sample chamber is configured with an inlet and outlet port and is configured to wash said first capture surface and, when present, said second capture surface, and deliver aerosol, and/or dust and/or ice particles, or components thereof to a chemical analysis system operably coupled to said sample chamber.

Embodiment 126: The particle capture system of embodiment 125, wherein said chamber is configured to deliver dust or ice particles to said chemical analysis system.

Embodiment 127: The particle capture chamber according to any one of embodiments 125-126, wherein said analysis system provides one or more analytic methods selected from the group consisting of optical microscopy, by optical spectroscopy, SEM, Raman spectroscopy, and mass spectrometry.

Embodiment 128: The particle capture chamber according to any one of embodiments 125-127, wherein said chemical analysis system comprise a programmable microfluidic analyzer (PMA) operably coupled to said capture chamber.

Embodiment 129: The particle capture chamber of embodiment 128, wherein said PMA comprises: a plurality of pneumatic inputs; a plurality of microfluidic channels; and a plurality of μCE separation channels, where said pneumatic inputs microfluidic channels and μCE separation channels are configured to so that fluid samples enter and leave the processor through access ports at the bottom; wherein an array of valves drive fluid routing on the PMA; and sample and reagent storage is provided in addressable wells at the top.

Embodiment 130: The particle capture chamber of embodiment 129, wherein said PMA permits analysis of different samples.

Embodiment 131: The particle capture chamber according to any one of embodiments 110-130, wherein said capture surface comprises a component in an aircraft, a rocket, a satellite or space probe.

Embodiment 132: A method of detecting organic compounds in high velocity dust, aerosol, and/or ice particles, said method comprising: providing a particle capture chamber according to any one of embodiments 98-108 in a high velocity particle plume where the lid of said particle capture chamber is open permitting particles comprising said plume to impact said first particle capture surface and, when present, said second particle capture surface to provide one or more surfaces with captured particles; closing the lid of said particle capture chamber to define a closed sample chamber; and analyzing said captured particles to identify presence and composition of organic molecules associated with said captured particles.

Embodiment 133: The method of embodiment 132, wherein said lid is open for at least a period of time sufficient to capture a detectable quantity of particles.

Embodiment 134: The method according to any one of embodiments 132-133, wherein said high velocity particle plume comprises an extraterrestrial particle plume.

Embodiment 135: The method of embodiment 134, wherein said high velocity particle plume comprises a particle plume at Europa or Enceladus.

Embodiment 136: The method according to any one of embodiments 132-133, wherein said particles comprise particles in a Venus cloud.

Embodiment 137: The method according to any one of embodiments 132-133, wherein said particles comprise comet debris.

Embodiment 138: The method according to any one of embodiments 132-133, wherein said particles comprise particles at high altitude.

Embodiment 139: The method according to any one of embodiments 132-133, wherein said particles comprise particles in low earth orbit.

Embodiment 140: The method according to any one of embodiments 132-139, wherein said analyzing comprises in situ analysis of said captured particles on said one or more capture surface(s).

Embodiment 141: The method of embodiment 140, wherein said in situ analysis comprises a spectroscopic analysis.

Embodiment 142: The method according to any one of embodiments 140-141, wherein said in situ analysis by one or more methods selected from the group consisting of SEM scanning, optical microscopy to identify absorption or emission of inorganic or organic materials, or detection of absorbance, fluorescence, phosphorescence or light scattering, or mass spectroscopy.

Embodiment 143: The method according to any one of embodiments 140-142, wherein said in situ analysis comprises Raman spectroscopy.

Embodiment 144: The method according to any one of embodiments 132-143, wherein said method comprises: warming said sample chamber if necessary; filling said sample chamber with a solvent or solvent system to suspend or dissolve organic molecules present on or in said particles; transporting the suspended or dissolved organic molecules into a microfluidic processor; and performing electrophoresis of said suspended or dissolved organic molecules in said microfluidic processor.

Embodiment 145: The method of embodiment 144, wherein said solvent or solvent system comprises water or a buffer.

Embodiment 146: The method of embodiment 144, wherein said solvent or solvent system comprises an aqueous two-phase partitioning system that partitions the analyte(s) (e.g., aerosol, and/or ice, and/or dust particles) or components thereof into one phase or into an interface between two phases comprising said partitioning system.

Embodiment 147: The method of embodiment 146, wherein said aqueous two-phase partitioning system comprises a system selected from the group consisting of oil/water systems, polymer/polymer systems, and polymer/salt systems.

Embodiment 148: The method according to any one of embodiments 146-147, wherein said two phase partitioning system comprises component 1 and component 2 as in Table 1.

Embodiment 149: The method of embodiment 148, wherein said two phase partitioning system comprises and oil/water system.

Embodiment 150: The method according to any one of embodiments 144-149, wherein said method comprises labeling one or more of amines, amino acids, carboxylic acids, aldehydes, ketones, and thiols with a fluorescent label.

Embodiment 151: The method according to any one of embodiments 144-150, wherein said electrophoresis comprises high-resolution capillary electrophoresis.

Embodiment 152: The method according to any one of embodiments 144-151, wherein said capillary electrophoresis comprises laser-induced fluorescence to detect the electrophoresed analytes.

Embodiment 153: The method according to any one of embodiments 144-152, wherein said capillary electrophoresis is performed by using a programmable microfluidic analyzer (PMA) operably coupled to said capture chamber.

Embodiment 154: The method of embodiment 153, wherein said PMA comprises: a plurality of pneumatic inputs; a plurality of microfluidic channels; and a plurality of μCE separation channels, where said pneumatic inputs microfluidic channels and μCE separation channels are configured so that fluid samples enter and leave the processor through access ports at the bottom; wherein an array of valves drive fluid routing on the PMA; and sample and reagent storage is provided in addressable wells at the top.

Embodiment 155: The method of embodiment 154, wherein said PMA permits analysis of different samples.

Embodiment 156: The method according to any one of embodiments 132-155, wherein said method provides data about any proteinogenic, biotic and abiotic amino acid that informs decisions about possible life.

Embodiment 157: The method according to any one of embodiments 132-156, wherein said method detects, identifies and quantifies one or more of Ala, Asp, Glu, Gly, His, Leu, Ser, Val, beta-Ala, GABA, Iva, and AIB.

Embodiment 158: The method of embodiment 157, wherein said method detects, identifies and quantifies Ala, Asp, Glu, Gly, His, Leu, Ser, Val, beta-Ala, GABA, Iva, and AIB.

Embodiment 159: The method according to any one of embodiments 132-158, wherein said method provides at least 2% quantitation relative to glycine with a sensitivity of 2 femtomoles of captured organic target material (10 nM in 180 micrograms of captured ice).

Embodiment 160: The method according to any one of embodiments 132-159, wherein said method provides chiral amino acid separations by running a test set consisting of histidine, alanine, serine and Asp and or Glu along with one abiotic amino acid such as Iva.

Embodiment 161: A method of detecting organic compounds in high velocity dust, aerosol, and/or ice particles, said method comprising: providing a particle capture chamber according to any one of embodiments 110-130 in a high velocity particle plume where the lid of said particle capture chamber is open permitting particles comprising said plume to impact said first particle capture surface and, when present, said second particle capture surface to provide one or more surfaces with captured particles; closing the lid of said particle capture chamber to define a closed sample chamber where closing said lid creates a reduced volume sample chamber defined by features on said first particle capture surface, and when present said second particle capture surface; and analyzing said captured particles to identify presence and composition of organic molecules associated with said captured particles.

Embodiment 162: The method of embodiment 161, wherein said lid is open for at least a period of time sufficient to capture a detectable quantity of particles.

Embodiment 163: The method according to any one of embodiments 161-162, wherein said high velocity particle plume comprises an extraterrestrial particle plume.

Embodiment 164: The method of embodiment 163, wherein said high velocity particle plume comprises a particle plume at Europa or Enceladus.

Embodiment 165: The method according to any one of embodiments 161-162, wherein said particles comprise particles in a Venus cloud.

Embodiment 166: The method according to any one of embodiments 161-162, wherein said particles comprise comet debris.

Embodiment 167: The method according to any one of embodiments 161-162, wherein said particles comprise particles at high altitude.

Embodiment 168: The method according to any one of embodiments 161-162, wherein said particles comprise particles in low earth orbit.

Embodiment 169: The method according to any one of embodiments 161-168, wherein said analyzing comprises in situ analysis of said captured particles on said one or more capture surface(s).

Embodiment 170: The method of embodiment 169, wherein said in situ analysis comprises mass spectroscopic analysis (e.g., laser adsorption mass spectrometry).

Embodiment 171: The method of embodiment 169, wherein said in situ analysis comprises a spectroscopic analysis.

Embodiment 172: The method according to any one of embodiments 169-171, wherein said in situ analysis one or more methods selected from the group consisting of SEM scanning, optical microscopy to identify absorption, light scattering or emission of inorganic or organic materials, or detection of fluorescence or phosphorescence.

Embodiment 173: The method according to any one of embodiments 169-172, wherein said in situ analysis comprises Raman spectroscopy.

Embodiment 174: The method according to any one of embodiments 161-173, wherein said method comprises: warming said sample chamber if necessary; filling said sample chamber with a solvent or solvent system to suspend or dissolve organic molecules present on or in said particles; transporting the suspended or dissolved organic molecules into a microfluidic processor; and performing electrophoresis of said suspended or dissolved organic molecules in said microfluidic processor.

Embodiment 175: The method of embodiment 174, wherein said solvent or solvent system comprises water or a buffer.

Embodiment 176: The method of embodiment 174, wherein said solvent or solvent system comprises an aqueous two-phase partitioning system that partitions the analyte(s) (e.g., aerosol, and/or ice, and/or dust particles) or components thereof into one phase or into an interface between two phases comprising said partitioning system.

Embodiment 177: The method of embodiment 176, wherein said aqueous two-phase partitioning system comprises a system selected from the group consisting of oil/water systems, polymer/polymer systems, and polymer/salt systems.

Embodiment 178: The method according to any one of embodiments 176-177, wherein said two phase partitioning system comprises component 1 and component 2 as in Table 1.

Embodiment 179: The method of embodiment 178, wherein said two phase partitioning system comprises and oil/water system.

Embodiment 180: The method according to any one of embodiments 174-179, wherein said filling said sample chamber with a solvent or solvent system comprises washing said one or more channels with a volume of less than about 100 μL, or less than about 75 μL, or less than about 50 μL, or less than about 40 μL, or less than about 30 μL, or less than about 20 μL, or less than about 15 μL of said solvent or solvent system.

Embodiment 181: The particle capture surface of embodiment 180, wherein said filling said sample chamber with a solvent or solvent system comprises washing said one or more channels with a volume as small as 10 μL or less.

Embodiment 182: The method according to any one of embodiments 180-181, wherein said volume is fluid volume smaller than the total volume of the chamber of said sample chamber without the microchannels present thereby concentrating said chemical/biochemical contents.

Embodiment 183: The particle capture chamber of embodiment 182, wherein the volume is less than 10%, or less than about 5%, or less than about 2% of the total volume of the chamber of said chamber without the microchannels present.

Embodiment 184: The particle capture chamber according to any one of embodiments 180-183, wherein the concentration of analyte in said extractant fluid is increased by at least at least 2-fold, or at least about 5-fold, or at least about 10-fold, or at least about 20-fold as compared to the concentration of said analyte present in a volume of extractant fluid equal to the total volume of said chamber.

Embodiment 185: The method according to any one of embodiments 174-181, wherein said method comprises labeling one or more of amines, amino acids, carboxylic acids, aldehydes, ketones, thiols, and polycyclic aromatic hydrocarbons (PAHs) with a fluorescent label.

Embodiment 186: The method according to any one of embodiments 174-185, wherein said electrophoresis comprises high-resolution capillary electrophoresis.

Embodiment 187: The method according to any one of embodiments 174-186, wherein said capillary electrophoresis comprises laser-induced fluorescence to detect the electrophoresed analytes.

Embodiment 188: The method according to any one of embodiments 174-187, wherein said capillary electrophoresis is performed by a programmable microfluidic analyzer (PMA) operably coupled to said capture chamber.

Embodiment 189: The method of embodiment 188, wherein said PMA comprises: a plurality of pneumatic inputs; a plurality of microfluidic channels; and a plurality of μCE separation channels, where said pneumatic inputs microfluidic channels and μCE separation channels are configured to so that fluid samples enter and leave the processor through access ports at the bottom; wherein an array of valves drive fluid routing on the PMA; and sample and reagent storage is provided in addressable wells at the top.

Embodiment 190: The method of embodiment 189, wherein said PMA permits analysis of different samples.

Embodiment 191: The method according to any one of embodiments 161-190, wherein said method provides data about any proteinogenic, biotic and abiotic amino acid that informs decisions about possible life.

Embodiment 192: The method according to any one of embodiments 161-191, wherein said method detects, identifies and quantifies one or more of Ala, Asp, Glu, Gly, His, Leu, Ser, Val, beta-Ala, GABA, Iva, and AIB.

Embodiment 193: The method of embodiment 192, wherein said method detects, identifies and quantifies Ala, Asp, Glu, Gly, His, Leu, Ser, Val, beta-Ala, GABA, Iva, and AIB.

Embodiment 194: The method according to any one of embodiments 161-193, wherein said method provides at least 2% quantitation relative to glycine with a sensitivity of 2 femtomoles of captured organic target material (10 nM in 180 micrograms of captured ice).

Embodiment 195: The method according to any one of embodiments 161-194, wherein said method provides chiral amino acid separations by running a test set consisting of histidine, alanine, serine and Asp and or Glu along with one abiotic amino acid such as Iva.

### Definitions

The following abbreviations are used herein: AA=amino acid, CE=Capillary Electrophoresis, EOA=Enceladus Organic Analyzer, LIF=Laser Induced Fluorescence, CELF=Capillary Electrophoresis Life Finder, PMA=Programmable Microfluidic Array, PAH=polycyclic aromatic hydrocarbons, Berkeley=University of California at Berkeley, SiPM=Silicon Photomultiplier, SSL=Space Sciences Laboratory at Berkeley.

The term “capture efficiency” refers to the percentage of particle materials that impact a particle capture surface that are effectively retained by that surface.

The term “organic degradation” refers to degradation of one or more organic compounds upon impact of a particle containing those organic compounds on a surface. In certain embodiments the organic degradation is due to heating on impact. Organic degradation can also be caused by the shock of the impact. Organic degradation is said to occur when the identity of the organic molecule(s) cannot be definitively ascertained by structurally sensitive analytic procedures such as Raman spectroscopy, mass spectrometry, capillary electrophoresis and the like.

When the terms “high velocity” or “high velocity particle plume” are used herein, the high velocity refers to the relative velocity between a capture surface and a set of particles being captured. Thus, the high velocity can be a consequence of capture surface movement in addition to or as an alternative to absolute particle velocity. High velocity typically refers to a velocity from 10 to 100's of m/s while hypervelocity refers to km/s velocities typically from 1 to 5 km/s or higher.

A “soft metal” or “soft metal alloy” refers to a metal or metal alloy with a Mohs hardness rating of less than about 3. Illustrative soft metals include, but need not be limited to lead, gold, silver, tin, zinc, indium, mercury, aluminum, copper, brass, and bronze.

Aqueous two-phase systems (“ATPS”) are biphasic systems composed two materials (e.g., an oil and an aqueous component) that can be used to partition analytes into one of the two phases or into an interface between the two phases and thereby separate and/or concentration the desired analyte(s). ATPS systems are traditionally formed by two polymers or one polymer and one salt. However, the classification currently includes, but is not limited to, ATPS formed by ethanol, micelles, ionic liquids, and the like.

### BRIEF DESCRIPTION OF THE DRAWINGS

FIG. 1. CAD showing CELF components. The labeled subsystems include Sample Capture system connected to Macrofluidics system; Microchip CE and PMA microprocessor manifold; redundant LIF Detection systems; water storage reservoirs and gas pressure system.

FIG. 2. Schematic of CELF biomarker analysis process showing dissolution of plume ice in the capture chamber, transport to the PMA for labeling of amines and amino acids, injection for high resolution CE separation, and quantitation and molecular analysis by fluorescence intensity and molecular mobility

FIG. 3. (Top) SEM image of NaCl residue (green) from an impact between a frozen brine projectile and an aluminum target with an impact velocity of 1.5 km/s. (Bottom) SEM image of PMMA residue (red) from an impact between a PMMA projectile and an indium target with an impact velocity of 1.9 km/s.

FIG. 4 Raman spectra of the captured PMMA material in FIG. 3 (top three traces) demonstrates that PMMA (characteristic pure sample Raman spectrum at the bottom) survives the impact and is captured in the crater and its surroundings.

FIG. 5. Plot of modeling results for simulated ice particle impacts into Au and Al as a function of input velocity.

FIG. 6. Panel A) Representative CAD of plume capture system showing the mechanics in a 90 degree open position for 45 degree plume impacts. The opening angle, total capture area, and capture material are determined by the mission science requirements and flight profile. The tan surfaces are the capture plates shown in more detail in panel B. Panel B) Illustrative design for a serpentine microfluidic channel structure that spans the inside diameter of a 100 mm diameter target. The inlet at the top provides a flow of water (or other solvent or solvent system) that proceeds back and forth in the channels finally exiting at the bottom hole that connects to the PMA and analyzer. In certain embodiments, the corresponding lid may be featured or unfeatured and different channel layouts used.

FIG. 7 shows one illustrative embodiment of a capture surface 01. The illustrated surface is configured with a serpentine channel 02 and entrance and exit ports into the serpentine channel 03 are shown.

FIG. 8 shows one illustrative, but non-limiting embodiment of a capture 01 surface with an associated lid 04 where the lid can comprise a second capture surface. The figure also illustrates an inlet/outlet port 06.

FIG. 9 shows some illustrative, but non-limiting microchannel cross-sections. The illustrated cross-sections include rectangular (A), a cross-section with chamfered sides (B), a cross-section with a curved bottom (C), and a cross-section with sloping sides (D). The arrows indicate possible trajectories of particles striking the microchannels. As illustrated in B, C, and D, some microparticles that impact beveled or curved portions of the channel can be reflected into another part of the channel.

FIG. 10 shows some illustrative, but non-limiting, designs of some of the ways to introduce channel features to control flow patterns over the capture chamber surface. Top: Control of flow pattern by flow input manifold. (Bottom left) spiral design. (Bottom right) Bifurcated channel design.

FIG. 11 shows some illustrative, but non-limiting serpentine channel patterns. A serpentine channel refers to a winding channel. In certain embodiments a serpentine channel increases the area of the surface in which the channel is disposed. That is, the surface area of the channel is greater than the projected surface area of the underlying surface. Illustrative serpentine channel patterns include, but are not limited to a square spiral serpentine channel (A), a circular spiral serpentine channel (B), a switchback serpentine channel (C), and the like.

FIG. 12 shows an illustrative, but non-limiting example of a particle capture surface comprising zones of different hardness.

FIG. 13 illustrates on embodiment of CELF instrument comprising pneumatic, macrofluidic, microfluidic and detection subsystems.

FIG. 14. Top: Layout of one embodiment of a 100 mm dia. organic analyzer microfluidic device including pneumatic inputs (blue), microfluidic channels (green), and μCE separation channels (red). Fluid samples enter and leave the processor through the four access ports at the bottom. The central square array of valves drive fluid routing on the chip. Sample and reagent storage occurs in the addressable wells at the top which in this configuration enable the analysis of six different samples. Bottom, manifold with fluid connections and pneumatic valves to control the chip.

FIG. 15. Photograph (top) and schematic (bottom) of the light gas gun (LGG) at the University of Kent. A shotgun cartridge detonates which drives a piston that compresses a light gas in the pump tube. A thin aluminum disc ruptures at a specific pressure (˜2 kBar) allowing the compressed gas to accelerate the projectile (housed in a sabot) through an evacuated launch tube. Light curtains and impact sensors are used to measure the velocity. The target is positioned in either the blast tank or target chamber—both of which are evacuated.

FIG. 16. SEM image of an indium capture surface after impacts with 4 μm diameter PMMA particles at a velocity of 0.547 km s⁻¹. Particles and dents are observed on the foil, with most particles exhibiting no detectable deformation and one with a minor fracture in the top of the image.

FIG. 17. (Top) SEM image of 4 μm (pre-impact) diameter PMMA particles stuck to the silver capture surface after a 0.851 km s⁻¹ velocity impact. A 7 μm scale bar has been positioned over one of the particles to highlight the resulting size deformation. (Bottom) SEM-EDX image of the silver capture surface foil after a 0.979 km s⁻¹ velocity impact with a 10 μm diameter particle that rebounded off the foil and deposited a ring of carbon-rich residue around the dent.

FIG. 18. SEM-EDX images of an impact crater and dent on indium (left) and copper (right) caused by impacts with 10 μm diameter PMMA particles at a velocity of 1.94 km s⁻¹. Well-defined stringy PMMA/carbon residue (top) and amorphous residue (bottom) is highlighted.

FIG. 19. SEM-EDX image of a crater from a 10 μm diameter PMMA particle on the indium capture surface after an impact at a velocity of 2.92 km s⁻¹. Well defined stringy PMMA/carbon residue is highlighted.

FIG. 20. Mean residue coverage (MRC) plotted against impact velocity for Ag, Al, Au, Cu, and In targets for each particle diameter (4, 6 and 10 μm). Error bars (±10%) represent the uncertainty in the residue area and sticking coefficient due to manual measurements using ImageJ.

FIG. 21. Raman spectra of pre-shot PMMA particles (top), a particle captured on the indium capture surface at 0.995 km s⁻¹ velocity (middle) and residue captured in a crater on the indium target at 1.94 km s⁻¹ velocity (bottom). The spectra indicate that captured PMMA remains chemically intact up to ˜2 km s⁻¹ velocity.

FIG. 22. Velocity-crater calibration plots for ice simulants (PMMA) impacting the various CS materials at velocities ranging 1.0-3.0 km s⁻¹. Trend lines are plotted for the different particle diameters and shaded regions represent the standard deviation (1σ, n=10) of the mean crater diameters.

FIG. 23. Crater-particle size calibration plots for ice simulants (PMMA) impacting Ag, Al, Au, Cu and In targets with 1, 2 and 3 km s⁻¹ velocity. Error bars represent the uncertainty derived from the mean standard deviation (1σ) across the 1.0-3.0 km s⁻¹ velocity range for each particle size (4, 6 and 10 μm).

FIG. 24. SEM image of a 10 μm diameter PMMA particle stuck to gold foil after a 0.979 km s⁻¹ velocity impact. Thermal deformation of the particle and extruded residue deposited on the foil surrounding the particle can be observed.

FIG. 25. Craters on Al, Au, Cu and In target after 1.734 km s⁻¹ impact (G0105191) with a frozen projectile of Pacific Blue dye and a cysteine-water solution (12.5:4000 μL).

FIG. 26. (Top) SEM/EDX image of a crater in aluminum foil (left) after a 1.734 km s⁻¹ impact with an ice particle 10 μm in diameter. (Bottom) Sulphur EDX of the same crater showing significant amino acid (cysteine) recovered from the crater.

FIG. 27. Fluorescence microscope images of a 1.7 km/s impact ice shot G0105191 into a gold target demonstrating the capture and survival of significant quantities of the organic tracer molecule Pacific Blue in a hypervelocity ice impact.

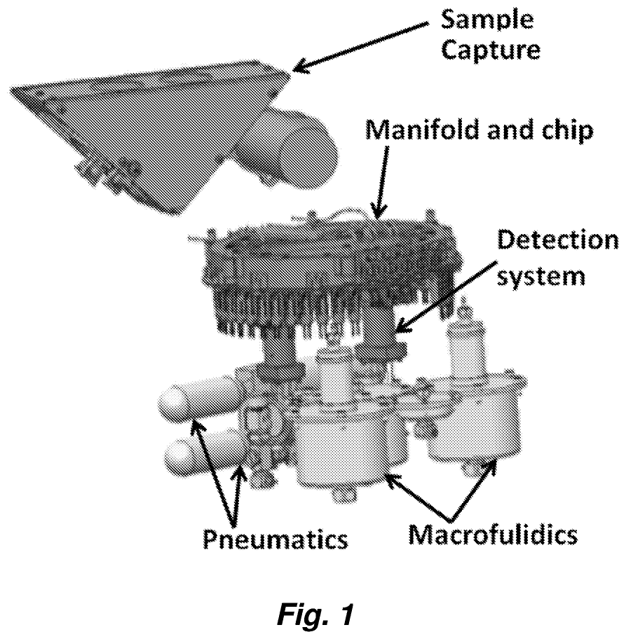

### DETAILED DESCRIPTION

In various embodiments a capture and analysis system is provided that efficiently captures high speed particles (e.g., high velocity dust, aerosol, and/or ice particles), does not degrade entrained organic molecules, provides for effective and efficient analysis of captured materials, can be readily cleaned to provide low background and forward contamination, and that has high sensitivity for analyzing trace organic molecules. In certain embodiments the capture and analysis system is effective for capturing high velocity extraterrestrial plume dust and ice particles, and/or for capturing high velocity particles in other environments, e.g., at high altitude, in near earth orbit, and the like.

CELF (Capillary Electrophoresis Life Finder) is a novel and rapidly maturing miniaturized microfluidic organic chemical and biochemical analyzer that, at high altitude, in outer atmosphere, or in outer space, can sensitively analyze cloud, dust and plume samples for organic molecules that may be indicative of past or present life. CELF addresses NASA Science Goals to “explore and find locations where life could have existed or could exist today” and provides science measurement capabilities for biosignatures that address the requirements for life detection suggested in the Europa Lander Study 2016 Report (SDT Report) (Hand et al. (2017) Report of the Europa Lander Science Definition Team. 1-264). CELF can probe for life signs by determining, inter alia, the abundances and patterns of organic biomarker compounds including, but not limited to, amines, amino acids and carboxylic acids with a sensitivity approaching 1 picomole per gram ice/particle sample. CELF can also determine the types, abundances and enantiomeric ratios of any amino acids in the sample. In one illustrative, but non-limiting embodiment, the miniaturized CELF instrument has size, mass (3.6 kg) and power requirements that can be accommodated in a variety of aircraft, high altitude balloon, rocket and space probe applications.

### Life Detection Approach

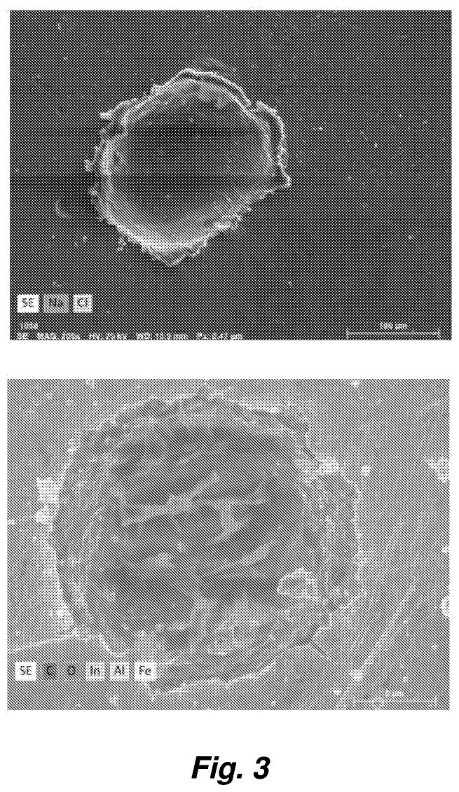

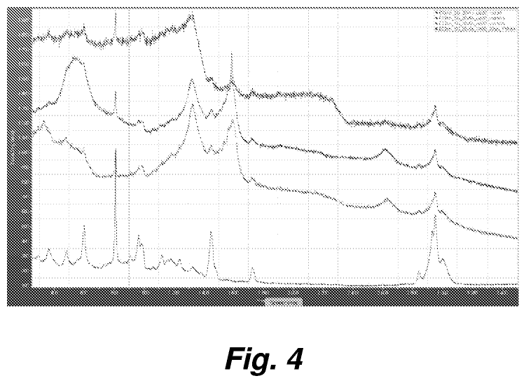

Using the unique capabilities of microchip capillary electrophoresis to look for molecular biomarkers indicative of extinct or extant life or that inform about the environment and habitability (Russell et al. (2014) Astrobiology, 14: 308-343) will produce information of lasting value about the chemistry and biochemistry of Europa (smallest of the four Galilean moons orbiting Jupiter) and many other locations in our solar system including Enceladus (the sixth largest moon of Saturn), comets, etc. Since the biologically available energy at Europa is 7-9 orders of magnitude below that of Earth (Hand et al. (2017) Report of the Europa Lander Science Definition Team. 1-264), microorganisms are expected at difficult to detect concentrations of only 0.1-100 cells/mL ((Id.), FIG. 3.4 therein). However, biological processes have the unique property of repetitively using subsets of molecular building blocks and/or combinatorial polymers for functional, energetic, structural and information processes. This process results in a focused amplification of molecules in biological cells that are not otherwise thermodynamically favored (see, e.g., Ehrenfreund et al. (2002) Rep. Prog. Physics, 65: 1427-1487; Eigenbrode (2008) Strategies of Life Detection, 2: 161-161; Georgiou & Deamer (2014) Astrobiology, 14: 541-549; Lovelock (1965) Nature, 207: 568-570; McKay (2004) PLOS Biology, 2: e302; FIG. 4.1.2 in Hand et al. (2017) Report of the Europa Lander Science Definition Team. 1-264). For example, a terrestrial bacterial cell contains roughly a billion amino acids but only 20 distinct amino acid side chains are utilized. In most cases, these amino acids are homochiral. Structural lipids and their head groups are also molecularly focused and amplified. Looking for biologically amplified chemical biomarkers makes detection at the low Europan concentrations more likely because there are billions of target molecules in each putative cell. The observation of molecular populations, focused by biosynthesis, including amino acid side chain identity, amino acid chirality, and carboxylic acid chain length variation, provides important information contributing to a life or no-life decision (see, e.g., Peters et al. (2005) The Biomarker Guide: Volume 1 Biomarkers and Isotopes in the Environment and Human History. Cambridge University Press; Summons et al. (2008) Space Science Reviews, 135(1-4): 133-159). These examples of biologically focused molecular distributions are useful to guide experimentation but the actual biological focusing on Europa may be significantly different from Earth. More generally, biologically focused molecular complexity (e.g., alkaloids, steroids, pigments) is also a powerful biosignature (Summons et al. (2008) Space Science Reviews, 135(1-4): 133-159) so any search for life should assess molecular complexity (Li & Eastgate (2015) Organic & Biomolecular Chem. 13: 7164-7176) through a large bandwidth analysis of molecular composition. For example, Cronin (Cronin & Walker (2017) Phil. Trans. R. Soc. A, 375: 20160342; Marshall et al. (2017) Phil. Trans. R. Soc. A, 375:20160342) has proposed and developed methods for scoring molecular complexity and determining thresholds that indicate biological processes. It is important to note that while we have developed microchip CE analysis methods for fluorescent labeled amino acids, the amine labeling methods will label and detect any organic molecules in the sample with a primary amino group (Chiesl et al. (2009) Anal. Chem. 81: 2537-2544) and the same is true for carboxylic acids (see, e.g. Stockton et al. (2011) Astrobiology, 11: 519-528, etc.). Microchip CE has the “bandwidth” needed to detect amino acids and carboxylic acids along with any other biomarkers that contain these important and ubiquitous functional groups. CELF is thus a broadly capable life detection instrument. It is also a broadly capable organic analyzer system that can be used to probe the organic content of clouds, plumes, debris fields etc. on earth and elsewhere in our solar system.

### Instrument Concept for Life Detection

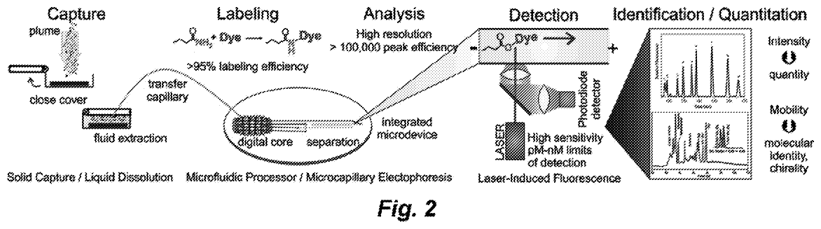

FIG. 2 presents a schematic of one illustrative embodiment of the CELF sample capture and analysis process, chip and instrumentation that will be detailed below. In certain embodiments, the plume sample is captured by an opened clam shell structure which is closed, warmed and then filled with an aqueous buffer to dissolve the amine, amino acid, and carboxylic acid molecules including biomarkers that are present in the sample. The dissolved analytes are then transported in a small volume of buffer into the CELF microfluidic processor where amines and amino acids are labeled with a very sensitive fluorescent dye. (Analogous labeling and detection methods are available for carboxylic acids, aldehydes, ketones and thiols, if desired, as well as polycyclic aromatic hydrocarbons or PAHs). In certain embodiments the labeled analytes are rapidly separated by high-resolution capillary electrophoresis in microfabricated channels followed by high sensitivity (e.g., 100 pM) laser-induced fluorescence (LIF) detection. The fluorescence intensity reveals the concentration of the biomarker while the molecular identity, a function of the charge and size of the analyte, is provided by the mobility or appearance time at the detector.

CELF builds upon 20 years of extensive lab and field studies at the University of California at Berkeley to develop microfabricated organic analyzers for planetary exploration, and leverages current NASA MatISSE funding to the Space Sciences Laboratory at the University of California at Berkeley (SSL) to mature the core microfabricated organic analyzer.

### Technical Approach

Europa Plume Capture System Design and Performance

Capturing an icy plume sample is an appealing way to obtain samples at Europa and/or Enceladus for example because the samples come from the most relevant under-ice ocean and pristine samples are presented without the technical and planetary protection problems of a surface lander. However, the amount of material that can be gathered is much less that the gram samples that would be provided by a lander (Sparks et al. (2016) Astrophys. J. 829: 121; Lorenz (2016) Icarus, 267: 217-219), and the encounter can occur at a relatively high velocity of 100 m/s to 5 km/s (depending on mission trajectory) making capture efficiency and organic degradation challenging. A capture and analysis system is needed that efficiently captures plume particles, that does not degrade the entrained organics, that can be effectively and efficiently analyzed, that can be readily cleaned to provide low background and forward contamination, and that has high sensitivity for analyzing the trace organics.

The ice plumes at Europa have not been studied as thoroughly as those at Enceladus so the density, particle size distribution, location and temporal fluctuations are not as well characterized (Id.). At a 10 km pass height, the largest particle size is 440 micron which is acceptable if these large particles have a low probability of encounter. The maximum in the particle size distribution is presumably smaller than 440 micron. Assuming a collector efficiency of 50% for ice particles and a practical collector area of 120 cm², these data predict the collection of 180 micrograms of ice in a transect of the Europa plume at 10 km pass height. The ice plumes at Enceladus are better characterized with particle sizes ranging from 1 to 4 microns. For a 10 cm² capture area, these plume densities predict the capture of 5 microgram of ice in our chamber. We conclude that with practical collector areas we can gather a reasonable amount of material for successful organic analysis (Skelley et al. (2005) Proc. Natl. Acad. Sci. USA, 102: 1041-1046; Kim et al. (2013) Anal. Chem., 85: 7682-7688; Stockton et al. (2009) Astrobiology, 9: 823-831). In addition, chemistries have been developed for the analysis of PAHs by CE (Stockton et al. (2009) Anal. Chem., 81, 790-796).

A fundamental problem that is solved by this invention is as follows. A large collector area is needed to capture as much of the plume molecules and micron-sized particles (dust or ice etc.) as possible. However this large area disadvantageously results in a large collector area to be interrogated and a large volume after the lid is closed. The impact surface must be chosen to capture the particles efficiently, for example by deforming to absorb energy and to capture the particle in the resulting crater. In particular, the impact surface should slow the particles down in the collision to minimize shock and thermal damage so that the organic materials are not degraded which would interfere with analysis of the particles and their contents. These desired properties will depend on the encounter velocity so the dependence of these properties on velocity must be understood and optimized. The capture surface is desirably capable of being cleaned to a very low organic background level so that low-level organics in the particles can be analyzed. For some analysis methods the captured particles can be analyzed directly on the capture surface. For other analysis methods, the captured materials can be washed off in a small volume of liquid solvent so that the dissolved analytes are minimally diluted thereby providing the highest analyte sensitivity for the chemical analyzer. Our capture system(s) achieve these goals with a unique method, apparatus, and process.

In various embodiments, the capture surface material is chosen to catch (and retain) the particles, to provide a compliant impact to reduce shock and thermal destruction, and to be readily cleaned and washed to remove the captured material for in situ analyses. We have thus chosen soft inert metals like Au, Ag, Cu, Al, In, alloys or laminates thereof, and the like for initial studies. These materials are readily fabricated (conventional machining, microfabrication, sputtering, electrodeposition) in a wide variety of configurations and thicknesses and can be cleaned to very low levels of contamination. These surfaces are also easily washed to remove captured ice particle residues for analysis when desired.

To establish the feasibility of the CELF approach, we have studied high and hypervelocity impacts between 0.5 and 5.0 km/s theoretically, as well as experimentally using the light gas gun at the University of Kent, UK. Thus far 5% brine ice, glass and PMMA particles have been explored, all of which exhibit significant levels of projectile capture in the aforementioned metals. An exemplary impact crater of a 5% brine ice particle into an aluminum target demonstrates chemical NaCl capture by this soft impact surface at 1.5 km/s, signifying the feasibility of this approach, as seen in FIG. 3 (top). We have also performed impact experiments between 0.4 km/s and 2.2 km/s with PMMA projectiles into soft Au, Ag, Cu, Al and In targets. As shown in FIG. 3 (bottom), a significant fraction of the PMMA projectile material is captured and survives intact, as indicated by the preservation of its characteristic Raman signature as shown in FIG. 4. Thus, this demonstrates organic capture and survival using our target materials at high and hyper velocity impacts and the fundamental concepts and feasibility of our approach.

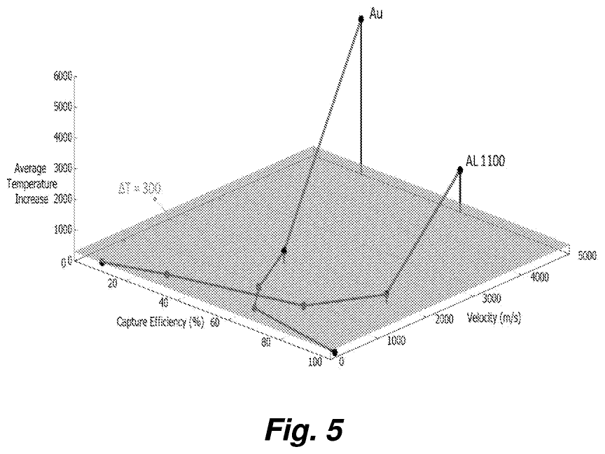

Valuable results have also been obtained by ANSYS Autodyn finite element analysis modeling of 2 μm ice simulant impacts into soft metals from 100 m/s to 5 km/s as summarized in FIG. 5. At low velocities (<1 km/s) we find that particles simply bounce off harder surfaces leaving no crater but they do deform and efficiently stick to softer surfaces. However, at higher velocities (1-3 km/s) the harder surfaces are more effective at capturing the incoming particles. For example, theoretical 3 km/s impacts into Al predict 80% capture efficiency. This behavior is illustrated in the figure where gold shows high capture efficiency at 100 m/s with a modest temperature increase but the capture efficiency falls as the velocity increases and the temperature increase is above 300 degrees above 2 km/s. On the other hand Al 1100 shows poor capture efficiency at velocities below 1 km/s where the particle just bounces off but good efficiencies and modest temperature increases through 2 km/s. These modeling results are consistent with the experimental impact studies showing survival of organic PMMA particle material at velocities of ˜2 km/s. We conclude that a soft metallic surface is a good choice for high velocity particle capture. In addition to pure metals it may be useful to fabricate multilayer metallic capture surfaces with a thin, approximately 1 to 5 micron thick, layer of a soft metal like gold or indium that can be vacuum or electrodeposited deposited onto a harder metal like silver or Al (e.g., Al 1100). This way the multi-layer surface captures both slow particles on the thin soft gold or indium surface and larger fast particles that penetrate the thin gold layer are efficiently captured in the Ag or Al layer. In one illustrative embodiment the capture surface comprises gold (Au) deposited on aluminum (e.g., Al 1100). In another illustrative, but non-limiting embodiment, the capture surface comprises gold deposited on silver.

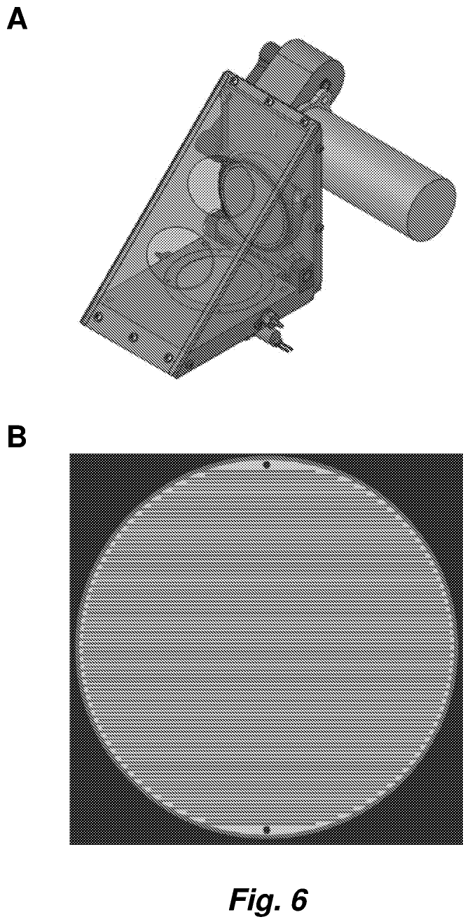

It is noted that additional layers can be added to fine tune this capture process and a variety of soft metal types and thicknesses can be used to optimize capture and chemical survival of the desired particles at desired velocities. In some cases it may be desirable to have the first capture surface tilted so the particles hit the target obliquely providing a longer impact distance and more extended deceleration. In this case it may also advantageous to place a second surface at an angle to the first surface to capture any ejecta or reflected particles from this primary collision as depicted in FIG. 6, panel A.

Once captured these captured particles and their contents can be analyzed by a variety of spectroscopies with no further processing. They could be scanned by SEM to determine elemental composition and the location of these elements relative to the crater as shown here in FIG. 3. They could be scanned by optical microscopy to look for the absorption or emission of inorganic or organic materials captured in the crater by fluorescence or phosphorescence. They could be scanned by Raman spectroscopy to look sensitively for the chemical contents of the particle in the crater as shown in FIG. 4. Methods for scanning such target or target material are described in the Europa Lander Study 2016 Report sections 4.5.3 Microscope for Life Detection (1).

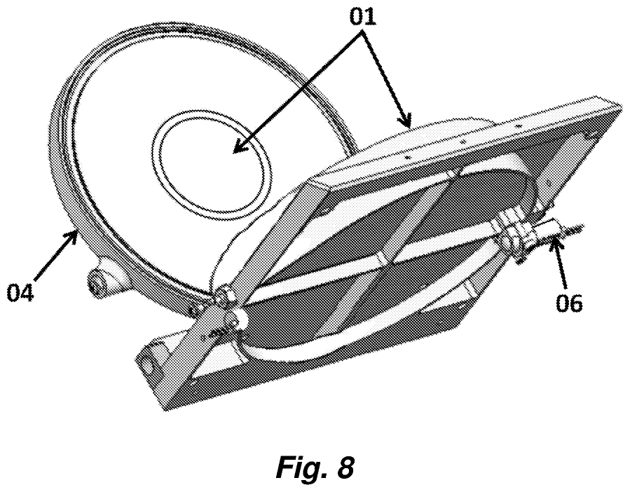

It is also desirable to dissolve the captured organic materials in the capture plate with the lowest possible volume of water buffer, or other solvent system, in order to minimize dilution of the captured analytes. The two capture surfaces in our chamber (see, e.g., FIG. 6, panel A) have a minimum gap of 100 micron between the two ˜100 mm dia. surfaces. This chamber is designed with a nominal 100 mm diameter wafer giving an effective capture area for normally incident particles of 120 cm² capture area but alternative larger and smaller designs can be used as determined by the plume density and the desired organic sensitivity. The capture can be performed on one surface using the other as a lid or the capture can be performed on both surfaces. One embodiment of such a device with a lid is illustrated in FIG. 8.

The impact angle can be 90 degrees, 45 degrees or other angles as desired. The second surface or lid is then closed on the target base defining an enclosed volume. This entire volume is then washed by pumping a solvent or buffer through the capture system transporting the dissolved analytes to the PMA. Fluid transport can be by conventional pumps, microfluidic pumps or using macroscopic pneumatic pressure sources defined below. The challenge with this design is dissolving the chemicals in the captured particles in the lowest possible solvent volume providing the highest possible concentration. It is important to keep the concentration of the analyte high since most analytical methods are concentration limited.

An expanded CAD of the two capture chamber surfaces and an illustrative, but non-limiting optimized fluidic transport system is shown in FIG. 6, panel B. In this design one of the two surfaces is structured to provide a serpentine folded network of channels that extend from the inlet port at one end of the wafer to the outlet port at the other end. The channels are on the order of 10-200 micron deep and can be from 20 to 400 micron in width but these detailed dimensions are not limiting and any serpentine or other folded or branched or spiral design for a channel that covers the majority of the surface area and passes fluidically from the input channel or channels to the output channel or channels can be utilized. Without these channels, the enclosed volume in a 100-micron deep and 100 mm diameter chamber would be on the order of 790 microliter. When the chamber is filled, e.g., with water, a buffer, or a 2-phase partition system for dissolution of target, the sample would be extremely dilute making detection difficult. On the other hand if a 10 microliter bolus of fluid was flowed down the serpentine channels, the concentration factor would be 790/10 or a factor of 80 concentration enhancement. Note that this 10 microliter volume is a large object in these microfluidic channels so it is easily manipulated. For example, if channel dimensions of 50 micron deep and 400 micron wide are used, 10 microliter is a 50 cm long bolus of fluid in these channels; such a bolus can be easily routed through tubing to the processor for analysis.

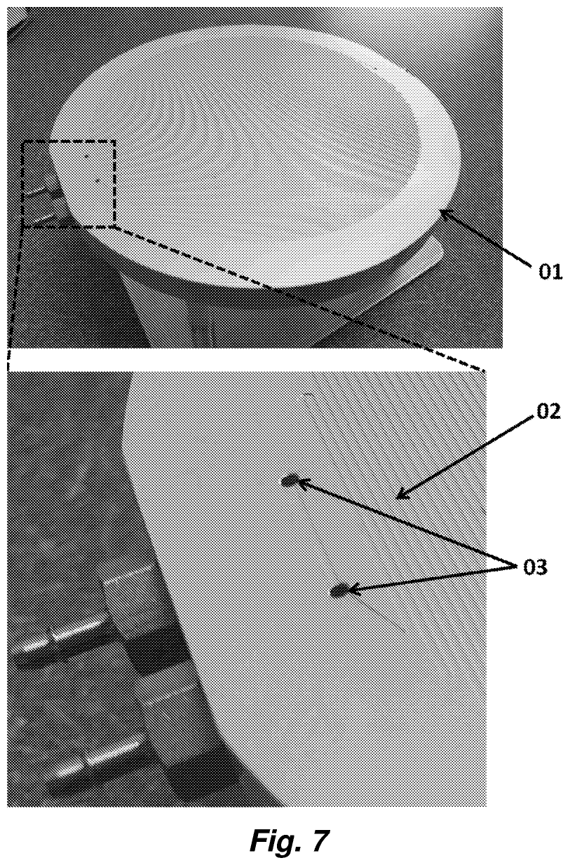

FIG. 7 shows one illustrative embodiment of a capture surface 01. The illustrated surface is configured with a serpentine channel 02 and ports into the serpentine channel 03 are shown. The illustrated surface comprises gold deposited on aluminum and the serpentine channel 02 was micromachined into the surface.

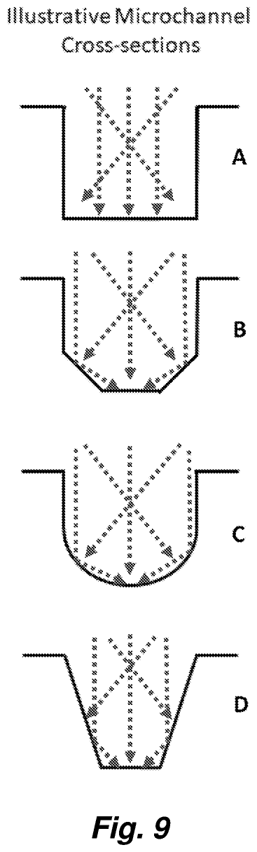

It will be noted that the microfluidic channels (e.g., serpentine channel) can have essentially any desirable cross-section. FIG. 9 shows some illustrative, but non-limiting microchannel cross-sections. The illustrated cross-sections include rectangular (A), a cross-section with chamfered sides (B), a cross-section with a curved bottom (C), and a cross-section with sloping sides (D). These different patterns can readily be determined by the choice of milling bit profile utilized when the channels are micromachined. The arrows indicate illustrative possible trajectories of particles striking the microchannels. As indicated by these arrows the cross-section of the microchannels can be selected so that the channel presents more surface normal (or close to normal) to particles striking at an angle other than 90 degrees to the bulk capture surface. Additionally, in certain embodiments, where the channels comprise beveled, or curved, or conical cross-sections, particles coming in at 90 degrees or normal to the overall surface will reflect off the conical, or chamfered, or beveled, or curved surface of the bottom of the channel and run into and be captured by the opposing wall of the channel since they will have lower velocity after initial impact, e.g., as illustrated in cross-sections “B”, “C”, and “D” in FIG. 9.

It will be recognized that the capture plate(s), e.g., capture plates that define a sample capture chamber, can have essentially any desired shape. In certain embodiments the projected area of the capture surface ranges from about 1 cm², or from about 5 cm², or from about 10 cm², or from about 20 cm², or from about 30 cm², or from about 40 cm², or about 50 cm², or from about 60 cm², or from about 70 cm², or from about 80 cm², or from about 90 cm², or from about 100 cm², up to about 1,000 cm², or up to about 500 cm², or up to about 400 cm², or up to about 300 cm², or up to about 200 cm², or up about 190 cm², or up to about 180 cm², or up to about 170 cm², or up to about 160 cm², or up to about 150 cm². In certain embodiments the projected area of the capture surface ranges from about 10 cm² up to about 200 cm², or from about 20 cm² up to about 150 cm², or from about 50 cm² up to about 120 cm².

In various embodiments capture surface can be essentially any shape that is convenient and/or desirable. In certain embodiments the shape can be irregular, an irregular polygon, or a regular polygon. In certain embodiments the shape of the projected area of said capture surface comprise a shape selected from the group consisting of circular, triangular, square, rectangular, and hexagonal. In certain embodiments the shape of the projected area of said capture surface is circular.

Using this system applied to the Europa example, our estimated limiting sensitivity with a 120 cm² capture area would be the detection of 10 picomoles of organics (e.g., amino acids)/gm ice sample for the 10 km pass. This can also be stated as a limiting detection of 10 nanomolar organics in the ice or a 1 ppb detection limit. The use of larger collector areas than 120-150 cm² to improve the detection limits further becomes mechanically impractical, for example hermetically sealing a larger chamber after capture would require prohibitive force to achieve reliable gasket sealing.

In various embodiments, we run a complete blank background run including washing the capture chamber with buffer followed by a complete analysis to determine the levels of any background organics in the instrument and/or reagents for comparison with the Europa sample. The pure metal capture surfaces themselves can be carefully and thoroughly cleaned and are not expected to get contaminated through adsorption (a significant problem for aerogels) or surface chemical reactions.

The flat capture surface can be fabricated by any of a number of conventional machining techniques for machining the harder materials such as Al, Ag, Cu. The softer metal layers Au or In for example can be produced by vacuum or sputter, or chemical, or electrochemical deposition onto a suitable harder machined substrate to the desired thickness (more than the incoming particle size) with a thin Cr adhesion layer if desired.

The featured substrate can be fabricated by more detailed but still standard techniques. First, the pattern can be fabricated by simply using a small end mill or its equivalent and milling out the channels in a flat Al, Cu or Ag substrate. Again if a softer metal surface is desired Au or In can be vacuum deposited onto the machined substrate through an appropriate mask. Note that it is possible to do electro-deposition of the softer metal target material onto a structured metallic substrate as well (see, e.g., Novak & Mathies (2013) Lab Chip, 13(8): 1468-1471). Alternatively the channel pattern could be fabricated by using an appropriate mask and chemically etching a metal substrate in the desired features (see, e.g., www.fotofab.com/wp-content/uploads/designguide-2017-milaero.pdf, and the like). This approach is useful when the channel depth is below 100-200 microns. This chemical etching can be performed on a variety of substrates including Al, Au and Cu. If alternative softer or multilayer metal surfaces are desired, a metal layer can be vacuum deposited or sputtered or electrodeposited onto the featured metallic surface (Vacuum Deposition” by Donald M. Mattox in Handbook of Physical Vapor Deposition (PVD) Processing (Second Edition) 2010, www.sciencedirect.com/topics/chemical-engineering/vacuum-deposition).

An alternative method of forming the fabricated surface is to etch the desired pattern in a glass wafer followed by metallic coating of the glass surface. Such glass fabrication and metallic coating is well known in the microfluidics field (Emrich & Mathies (2008) Microfabricated Electrophoresis Devices for High-Throughput Genetic Analysis: Milestones and Challenges, in Handbook of Capillary and Microchip Electrophoresis and Associated Microtechniques, ed. J. P. Landers, Third Edition (CRC Press), pp. 1277-1295, and references cited therein). In this approach the channel pattern is etched in the glass substrate and then coated with the desired target metal to the desired thickness using typically a Cr adhesion layer. The details of the channel pattern is not unique as long as the channels cover much of the substrate surface and seal effectively onto the lid when the system is closed for fluid flow.

Once closed, the two faces of the capture system must contact each other sufficiently well that the fluid flows sequentially down the length of the channel structure. This can be achieved with sufficiently flat substrates if they contact each other without gaps. The gaps may be better sealed by using a thin coating of a more compliant metal like indium deposited as a topcoat to form a seal between the top and bottom wafers. Another alternative is to treat the raised surfaces of the featured substrate with a hydrophobic barrier using microcontact printing (Ruiz & Chen (2007) Soft Matter, 3: 168-177) that prevents wetting in the thin gap between the featured and the top wafers. This can be done for example by providing a thin gold overcoat and treating the raised surfaces with a hydrophobic thiol reagent to increase the contact angle by standard methods (Sigma-Aldrich Product Information Technical Bulletin AL-266, “Preparing Self-Assembled Monolayers (SAMs): A Step-by-Step Guide for Solution Based Self-Assembly and references cited therein). Any microcontact printing method or chemistry that introduces a hydrophobic surface on the raised edges of the channels or that produces an effective gasket seal will be appropriate.

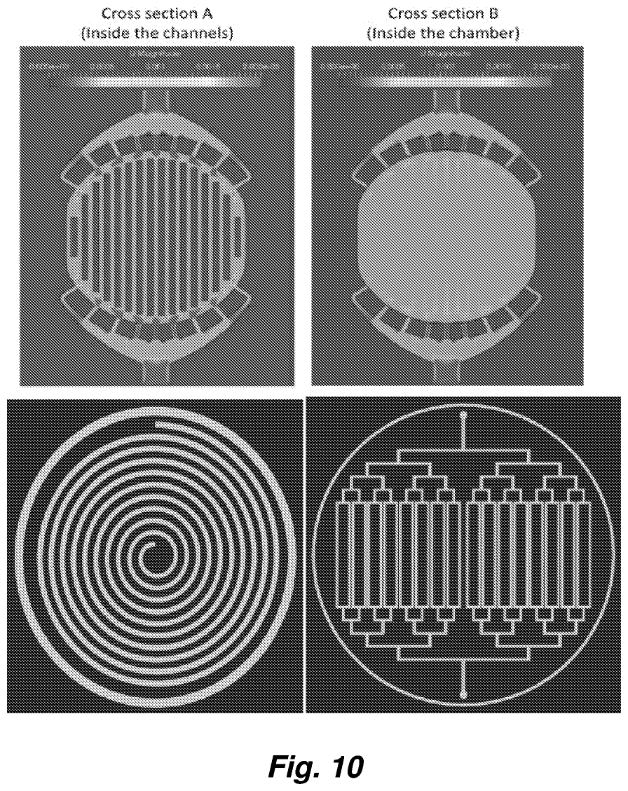

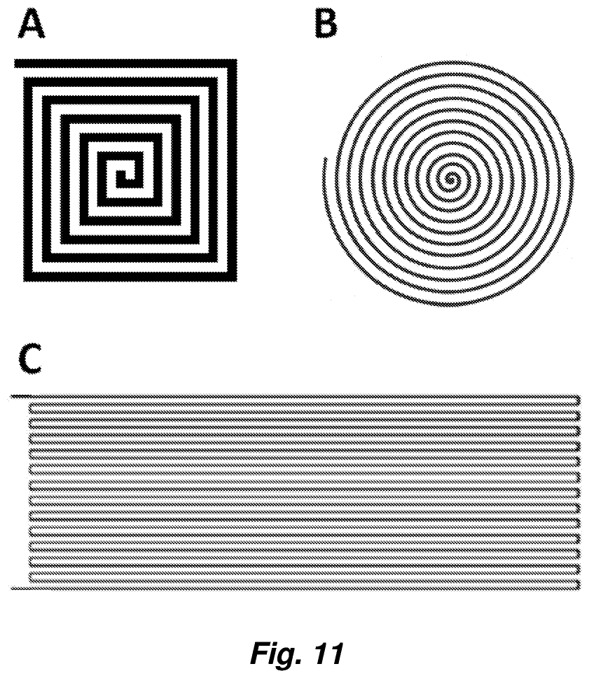

There are a number of ways to control the flow of extractant fluid through the closed capture chamber to optimize the dissolution of the captured material by dissolving it in a smaller solvent volume. An illustrative, but non-limiting, second design shown below FIG. 10 (top) uses an input and output manifold to provide a uniform flow profile through the chamber so that the leading front of the solvent dissolved the highest concentration analyte for detection. These structures can be fabricated by the methods described above. In certain embodiments, one could also use a concentric spiral design where the fluid comes in at the end or the start of the spiral channel (see, e.g., FIG. 11). In certain embodiments, one could also design a set of bifurcated channels at the inlet and at the outlet to manage this flow profile and achieve the same results. The foregoing channel designs are illustrative and not limiting. Any of a multiplicity of channel designs can be used to achieve the goal of producing a high concentration of analyte in the smallest possible extractant fluid volume.

Capture Surface Variations.

In certain embodiments the material composition of the various capture surfaces described above is substantially uniform. In such embodiments the thickness(es) of various layers comprising the capture surface and/or the material composition is substantially unchanged thereby providing a substantially constant hardness across the full area of the surface.

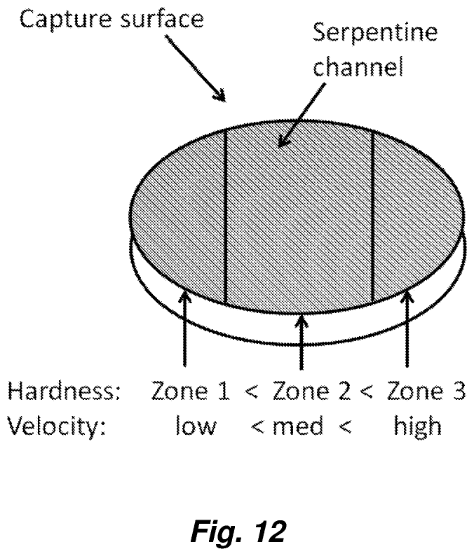

However, in certain embodiments, capture surfaces with different material layer thicknesses and/or different material composition in different regions are contemplated. Such capture surfaces can thereby provide different hardnesses in different regions of the surface. Thus, for example, as schematically illustrated in FIG. 12, different regions of the capture surface can present different hardnesses and thereby be adapted to optimize capture of participles of different velocities in each region. While, in certain embodiments, the capture surface can present different hardnesses in discrete zones as illustrated in FIG. 12, it will also be recognized that the zones need not be discrete. Thus, for example, in certain embodiments, capture surfaces are contemplated that present a gradient of hardness across the surface.

In certain embodiments, where microchannels are provided, a single microchannel can pass through all of the various hardness regions. However, in other embodiments, the surface can comprise different microfluidic channels in different hardness regions to provided independent collection and concentration of analytes from different velocity particles. Thus, by way of illustration, in certain embodiments, a single serpentine channel can pass through zones 1, 2, and 3 illustrated in FIG. 12. In other embodiments, each of zones 1, 2, and 3 can be serviced by a separate serpentine channel.

Wash/Extraction Systems.

In certain embodiments, where the chamber lacks microchannels, the entire chamber can be rinsed with a wash system to extract the aerosol, ice, and/or dust particles, or components thereof. Similarly, where microchannels are provided a bolus of fluid (wash system) can be pushed through the microchannels to extract and concentrate the aerosol, ice, and/or dust particles, or components thereof. In certain embodiments such “wash systems” comprise water and/or a buffer.

However, in certain embodiments, where further concentration of the aerosol, ice, and/or dust particles, or components thereof, the “wash system” can comprise an aqueous two-phase partitioning system. In certain embodiments such partitioning systems comprise water and a substantially immiscible (e.g., hydrophobic) component such as an oil. When washed with such a system the aerosol, ice, and/or dust particles, or components thereof can partition into one of the components comprising the two-phase system or into an interface between the two phases thereby effectively concentrating the analyte(s).

In various embodiments any of a number of aqueous two-phase (ATPS) systems are contemplated. Two-phase extraction/concentration systems are well known to those of skill in the art and include, but are not limited to oil/water systems, polymer/polymer systems, and polymer/salt systems. Illustrative, but non-limiting examples of two-phase partitioning systems are shown in Table 1.

TABLE 1
Illustrative aqueous two-phase extraction systems.

| | |
|---|---|
| Component 1 | Component 2 |

Oil/Water Systems

| | |
|---|---|
| Mineral oil | water |

Polymer/polymer Systems

| | |
|---|---|
| Polyethylene glycol | Dextran |
| | Ficoll |
| | Polyvinyl pyrrolidone |
| | Polyvinyl alcohol |
| | Hydroxypropyl starch |
| Polypropylene glycol | Dextran |
| | Hydroxypropyl dextran |
| | Polyvinyl pyrrolidone |
| Polyvinyl alcohol | Dextran |
| | Hydroxypropyl dextran |
| Polyvinyl pyrrolidone | Dextran |
| | Maltodextrin |
| Methyl cellulose | Dextran |
| | Hydroxypropyl dextran |
| Ethylhydroxyethyl cellulose | Dextran |

Polymer/salt Systems

| | |
|---|---|
| Polyethylene glycol | Potassium phosphate |
| | Sodium sulfate |
| | Magnesium sulfate |
| | Ammonium sulfate |
| | Sodium citrate |
| Propylene glycol | Potassium phosphate |
| Methoxypolyethylene glycol | Potassium phosphate |
| Polyvinyl pyrrolidone | Potassium phosphate |

In certain embodiments such two-phase systems can be used for more effective cleaning of the capture surface(s) between sample collection runs. It is also noted that two-phase partitioning systems have been implemented in microfluidic devices (see, e.g., Zhou et al. (2017) Lab Chip, 17: 3310-3317, and the like).

CELF System Design

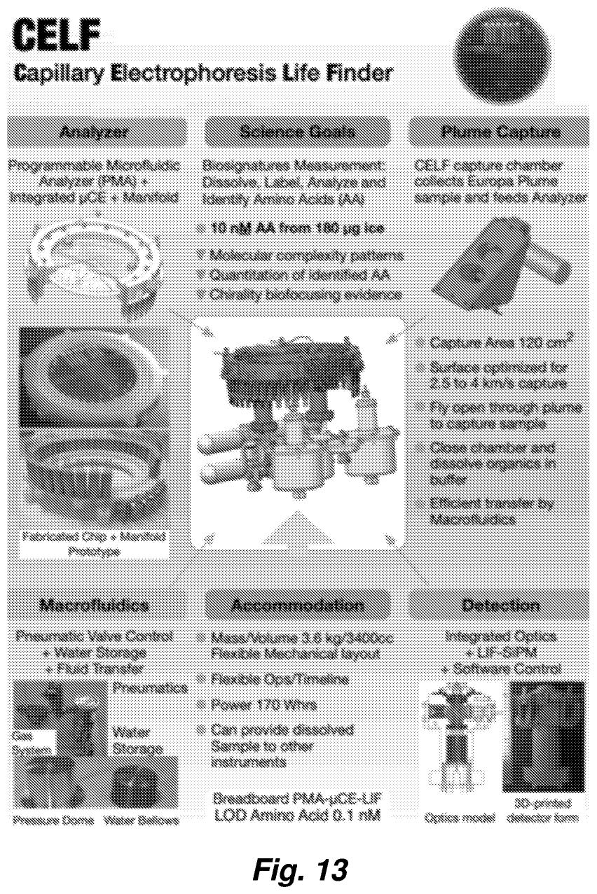

In various embodiments CELF can comprise pneumatic, macrofluidic, microfluidic and detection subsystems that are presented in FIG. 13 showing their relationships. Fluids are moved and controlled with pneumatics driven by a set of regulated nitrogen gas pressures. One set of pressures, routed by the solenoids on the manifold, control the pneumatic microvalves integrated into the PMA on the chip (see below). The nitrogen gas pressures are also used to route fluids in the macroscopic plumbing that connects the chip to the capture plate, waste reservoir, and fluid reservoirs. Gas pressure is used to move fluids in the connection tubing and to empty or fill the macroscopic reservoirs that consist of SS bellows.

The LIF confocal detection system CAD is also presented in FIG. 13. In certain embodiments, two redundant CE and detection systems are a part of this design. The fully integrated confocal module consisting of, for example, a diode laser and solid-state photomultiplier is currently under construction at SSL using flight capable components.

Microfluidic Chip Operation

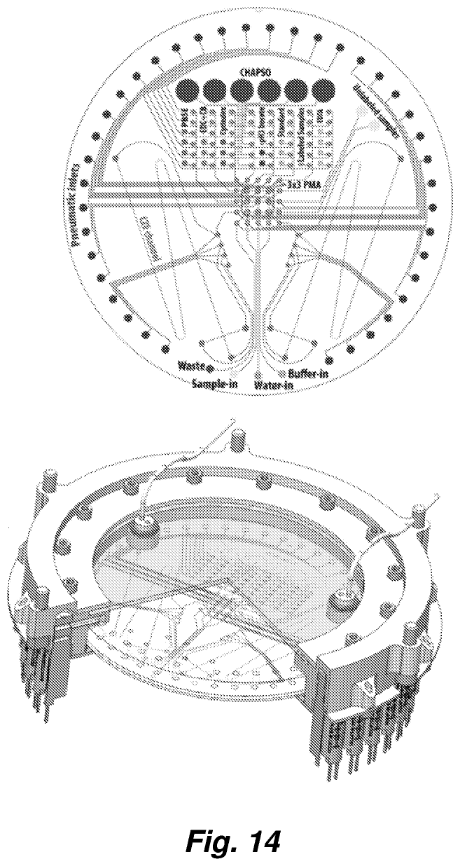

Central to the chip system is the Programmable Microfluidic Analyzer (PMA), which is an array of valves to route and mix fluids. FIG. 14 shows the layout for the multilayer microfluidic chip and manifold. Water is brought in from the water storage reservoirs and used to reconstitute/dissolve the various reagents stored dry in reagent wells. Sample is pumped into the chip through the Sample-In port and mixed with desired buffer and labeling reagent (Pacific Blue for amines; Cascade Blue for carboxylic acids) and allowed to react. The CE channel is then filled with buffer by the microfluidic pumps and the labeled sample is transported to the sample well of the injection cross for loading. Electric fields are applied to drive sample into the intersection of the red CE channels and then high voltage is switched on to drive the separation of molecules sweeping them toward the LIF detection point. The analyses for carboxylic acids and for amino acid chirality follow an analogous protocol all of which have been published in detail (Chiesl et al. (2009) Anal. Chem. 81: 2537-2544; Stockton et al. (2011) Astrobiology, 11: 519-528; Kim et al. (2013) Anal. Chem., 85: 7682-7688).

Detection and Analysis Validation

The detection and analysis requirements of the CELF instrument have been chosen so that they provide data about any proteinogenic, biotic and abiotic AA that inform decisions about possible life. During development, two Europa analog sets of AA will be examined. Detection, identification and quantification tests and optimization will be performed with the set (Ala, Asp, Glu, Gly, His, Leu, Ser, Val, β-Ala, GABA, Iva, AIB) with a goal of 2% quantitation relative to glycine with a sensitivity of 2 femtomoles of captured organic target material (10 nM in 180 microgram of captured ice). This test set includes 8 proteinogenic AA, 2 biotic AA and two AA that are only found abiotically. Separations will be optimized initially using amino acids labeled at micromolar concentrations; final optimization of labeling conditions and determination of the LOD can be performed using analyte concentrations in ice down to 10 nM. The possible impact of saline ices (including salts and divalent cations) on sensitivity and resolution will be explored using the methods we developed earlier using EDTA and effective borate buffers that ameliorate the effects of divalent cations and acid/base in the sample (Stockton et al. (2009) Astrobiology, 9: 823-831).

Chiral AA separations can be evaluated and optimized by running a test set consisting of histidine, alanine, serine and Asp and or Glu along with one abiotic amino acid such as Iva. These separations can be performed using the optimized mobile phase buffer, pseudo stationary phase, and temperature conditions determined above. Conditions can be developed that meet this criterion for chiral resolution while also meeting the amino acid detection and quantitation requirements outlined above.

This optimization of on-chip separations has a high likelihood of success because our previous on-chip work has demonstrated resolution of over 45 different amine and amino acid components in a single 2-minute separation (Chiesl et al. (2009) Anal. Chem. 81: 2537-2544). Previous work has also demonstrated multiple LIF confocal optical systems that deliver sensitivities of better than 100 picomolar. A system producing similar sensitivities has been developed at SSL and a flight capable version of this detection system is currently being fabricated.

### Conclusions

To gain new information about the chemistry and biochemistry of our solar system, it is essential to perform innovative measurements with innovative instruments. Habitability, biochemistry and life are fundamentally liquid water based processes. Wet chemical analysis methods are much better at probing these processes with high sensitivity and resolution compared to the traditional GCMS approaches that NASA has used in the past with limited success. The challenge and hence the innovation is developing instruments that can gather samples from clouds, dust, plumes etc. in high velocity transects and then perform high performance liquid-phase analyses within the constraints of a space mission. The capture chamber system disclosed here coupled with the microfluidic chemical/biochemical analysis systems can meet this challenge, and will revolutionize our search for enhanced chemical/biochemical understanding of our planet and our solar system.

### Example 1

### Characterizing Organic Particle Hypervelocity Impacts on Inert Metal Surfaces: Foundations for Capturing Organic Molecules in Hypervelocity Transits of Enceladus Plumes

### Summary.

The presence and accessibility of a sub-ice-surface saline ocean at Enceladus, together with geothermal activity, make it a compelling location to conduct further, in-depth, astrobiological investigations to probe for organic molecules indicative of extraterrestrial life. Cryovolcanic plumes in the south polar region of Enceladus enable the use of remote in situ sampling and analysis techniques. However, efficient plume sampling and the transportation of captured organic materials to an organic analyzer present unique challenges for an Enceladus mission. A systematic study, accelerating organic ice-particle simulants into soft inert metal targets at velocities ranging 0.5-3.0 km s⁻¹, was carried out using a light gas gun to explore the efficacy of a plume capture instrument. Capture efficiency varied for different metal targets as a function of impact velocity and particle size. Importantly organic chemical compounds remained chemically intact in particles captured at speeds up to 2 km/s. Calibration plots relating the velocity, crater- and particle-size were established to facilitate future ice-particle impact experiments where the size of individual ice particles is unknown

### INTRODUCTION

Studying the organic history, habitability and potential for, or presence of, extinct or extant life on any solar system body is an exciting, but challenging, quest. Instruments that can capture materials from plumes, clouds, comae or ejecta and perform sensitive analyses for organic molecules advantageously avoid the technical and planetary protection problems of a surface lander. In this regard the ice plumes emanating from Enceladus have recently attracted a great deal of attention. We experimentally explore the feasibility of hypervelocity organic particle capture using a series of capture surfaces. This work forms part of a project developing the Enceladus Organic Analyzer (EOA) instrument for probing biosignatures in icy plumes (Mathies et al. 2017).

The Cassini mission revealed a number of distinct, narrow geysers (Waite et al. 2006 and Porco et al. 2014) venting from four prominent, and warm, fractures in Enceladus' south polar region that make Enceladus a compelling target for noncontact analysis. These geysers form plumes that extend thousands of kilometers into space and are responsible for Saturn's E-ring (Spahn et al. 2006). Cassini data (e.g. Postberg et al. 2011), and ground-based telescope analysis of Saturn's E-ring (Schneider et al. 2009) suggests that the largest particles near the surface of Enceladus (2-6 μm diameter at an altitude of 50 km) are frozen droplets of salty liquid water (0.5-2.0% NaCl by mass) and that vapor in the plume includes trace amounts of ammonia and light organic compounds (Waite et al. 2009). Furthermore, a recent study reports observations of emitted ice grains containing concentrated and complex macromolecular organic material with molecular masses above 200 atomic mass units (Postberg et al. 2018). Various sources of evidence indicate that the plume originates from a sub-ice-surface liquid water ocean, with salinity similar to oceans on Earth, that is in contact with a rocky core (Postberg et al. 2009). Predictive models (Zolotov et al. 2007) and analysis of E-ring particles (Hsu et al. 2015) indicate hydrothermal sources at the ocean-core boundary, similar to the hydrothermal vents at Lost City (Kelley et al. 2005) on the Earth. The presence and accessibility of the salty liquid ocean, together with geothermal activity on Enceladus, make it the most promising place to conduct further, in-depth, astrobiological investigations using remote in situ analysis techniques to probe for organic molecules indicative of extraterrestrial life.

A variety of in situ instruments have been developed for probing organic molecules and biosignatures in planetary science, particularly probing the environment of Mars. The Sample Analysis at Mars (SAM) instrument uses Gas Chromatography-Mass Spectrometry (GCMS) to measure light isotopes (H, O, C, N), volatiles and to search for organic compounds directly from the atmosphere and any thermally released from solid samples (Mahaffy et al. 2012). The Mars Organic Molecule Analyzer (MOMA) onboard the ExoMars 2020 Rosalind Franklin rover will use GCMS and Laser Desorption Mass Spectrometry (LDMS) for organic analysis (Goesmann et al. 2017). The Scanning Habitable Environments with Raman and Luminescence for Organics and Chemicals (SHERLOC) instrument onboard the NASA Mars 2020 rover will use a deep ultra-violet Raman and fluorescence spectrometer that can characterize organic materials and will attempt to assess habitability and search for potential biosignatures (Beegle et al. 2016).

The EOA instrument (homepage located at eoa.ssl.berkeley.edu as of October 2019) currently in development at UC Berkeley Space Sciences Laboratory (SSL) is focused on the engineering of microfluidic chemical analysis flight systems with very high organic sensitivity and specificity based on the technology first developed, optimized and field tested in the Mars Organic Analyzer (Skelley et al. 2005; Skelley et al. 2007; Chiesl et al. 2009). For in situ studies of Enceladus, this technology has the advantages of small mass and size, autonomous operation, including fluidic manipulation, and high sensitivity for a variety of organic molecules that could be indicative of biosignatures. However, the requirement for efficient plume sampling and transport of the captured organic materials to the organic analyzer present unique challenges for an Enceladus mission.

Depending on the orbital navigation (e.g., a Saturn or Enceladus orbit), plume encounter speeds could range from a few hundred m s⁻¹ to several km s⁻¹, and fall into the ‘hypervelocity’ regime. During hypervelocity impacts both projectile and target undergo significant disruption and/or modification (for example, Avdellidou et al. 2015 and Wickham-Eade et al. 2018). This is due to shock pressures of the order of GPa (depending on the impact speed) and high temperatures (that can reach thousands of K, albeit for a brief amount of time) that are indicative of such impacts (Melosh 1989). Organic compounds are sensitive to both excessive shock and prolonged heating, both of which can alter bonds (Goldman et al. 2010), and thus the physical conditions that occur during an impact must be carefully considered when designing an organic material capture system.

A number of studies demonstrate the capture and survival of organic compounds during hypervelocity impacts. For example, Burchell et al. 2014 observed successful transfer of organic compounds from projectile to target during hypervelocity impacts: a frozen mixture of anthracene and stearic acid, solvated in dimethyl sulfoxide, was accelerated into targets of water ice, water and sand at velocities of ˜2 and ˜4 km s⁻¹. Price et al. 2012 explored the creation, destruction and modification of organic species during hypervelocity impacts of polystyrene particles, and this study found that Raman signatures of residual polystyrene were observed in the majority of craters after ˜1.5 and ˜6.1 km s⁻¹ impacts, but at higher speeds the majority of residue was elemental carbon. Parnell et al. 2010 successfully demonstrated the survival of organic biomarkers in craters and ejecta after hypervelocity impacts, by accelerating projectiles of stainless steel and siltstone to velocities ranging between 2-6 km s⁻¹ into targets of rock, water and sand. NASA's Stardust mission (Brownlee et al. 1997, 2006) exposed a collector made of silica aerogel and aluminum foil during passage through the tail of Comet Wild-2 at an encounter velocity of 6.1 km s⁻¹. Detection of organic material (Glavin et al. 2008 and Clemett et al. 2010) was observed inside several of the impact tracks on the aerogel collector. Although first thought to be potentially of terrestrial origin (Glavin et al. 2008), cometary glycine was found on the comet-exposed aluminum foils (Elsila et al. 2009), which is a more representative material of those expected on an Enceladus capture mission. Furthermore, polycyclic aromatic hydrocarbons (PAHs), most likely of cometary origin, that were unambiguously associated with impact residues were also identified on the foils (Leitner et al. 2008). This latter observation would lead us to suggest that a speed of ˜6 km s⁻¹ is the very highest limit for detectable survival of organic compounds onto a metallic (aluminum) collector.

These results demonstrate that organic molecules do survive hypervelocity impacts, but the extent of (unmodified) particle capture is unquantified. Although the amount of surviving impactor residue decreases as a function of impact velocity, at speeds of 6 km s⁻¹ the quantity of surviving residue becomes extremely difficult to detect (Kearsley et al. 2010), and the degree of residue alteration is also unknown. Furthermore, the relationship between organic survival (and/or alteration), capture efficiency, impact velocity and capture medium are not well defined, indicating the critical need for a detailed experimental study of these parameters.

To address this problem, we performed a systematic study where organic ice-particle simulants, with diameters of 4, 6 and 10 μm, were accelerated into soft inert metals at velocities of ˜0.5, 1.0, 2.0 and 3.0 km s⁻¹ with the objective of answering four fundamental questions: (1) How does the nature of impacts change with respect to velocity and target material? (2) What is the optimal material for effective particle capture during the specified impact velocities? (3) Do organic ice-particle simulants remain chemically intact during the impacts? (4) What is the projectile-crater size relationship at the specified impact velocities for the target materials?

### Experiments & Methodology

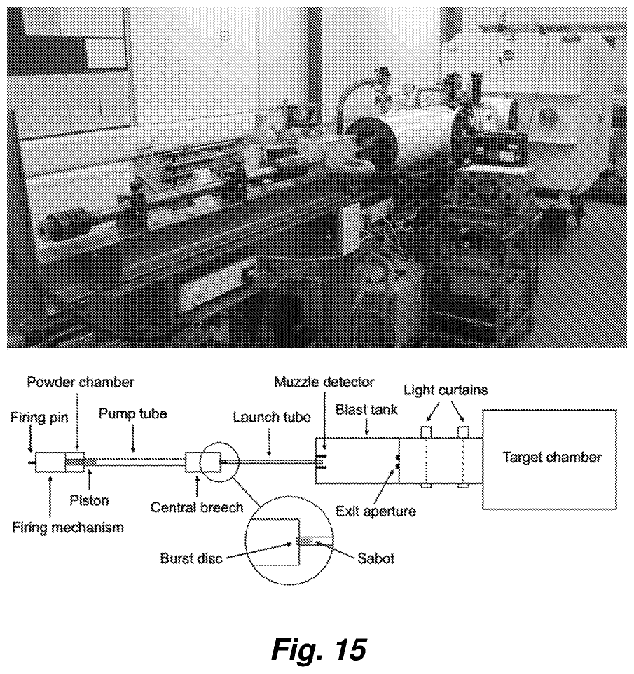

A series of high-velocity and hypervelocity impact experiments were carried out using the light gas gun (LGG) at the University of Kent (described by Burchell et al. 1999 and Hibbert et al. 2017), as seen and illustrated in FIG. 15. This particular LGG facility was selected due to its ability to accelerate a wide range of particles, both size and composition, up to velocities of ˜7.5 km s⁻¹ and offers flexibility in target configuration and temperature. Most critically, however, the LGG at Kent offers the unique capability of accelerating ice projectiles, which will be necessary for the second stage of this research and the EOA development.

Targets were designed to study the capture surface (CS) materials of interest. These materials were chosen to provide a compliant impact surface that can effectively capture particles and reduce shock and thermal destruction of the organic compounds in the projectiles. Additionally, the materials had to permit fabrication in a variety of configurations and thicknesses, suitable for space-flight instrument deployment. Finally, they had to be readily cleaned to very low levels of organic contamination and easily washed to extract captured particle residue for analysis. Thus, soft, relatively inert metal foils were chosen for test, that included aluminum (AL000630/11), copper (CU130118), gold (AU000345/104), indium (IN000260/15) and silver (AG000305/4). Five foils, one of each material, were configured in a 3/2 grid measuring ˜2×2 cm and were adhered to a carbon pad attached to a 6 mm thick aluminum disc. This design acted as a control allowing the projectiles to impact each target metal under nominally similar impact conditions for direct comparison.

Polymethylmethacrylate (PMMA) particles were selected as the ice-particle simulants for the experiments as they are solid at room temperature and have an accessible melting point (160° C.) and density (1.18 g cm⁻¹) similar to ice. The majority of the Enceladus plume mass, at an altitude of 50 km, resides in particles with a diameter of 4-6 μm (Hedman et al. 2009) and monodisperse PMMA particles of this size are readily available. Particles with diameters of 4 and 6 μm were selected to simulate plume particles, while additional 10 μm particles were included to extend the dataset. Monodisperse PMMA particles were selected in order to develop accurate particle-crater size calibration plots for the specified impact velocities on the different target materials. Finally, the mechanical profiles of PMMA polymers are well defined enabling the development of hypervelocity impact hydrocode models.

During each experiment, thousands of projectiles, either 4, 6 or 10 μm diameter were loaded into a sabot and fired onto the metal targets. The sabot was discarded in flight, leaving the projectiles to proceed to the target. The velocities of the projectiles (˜0.5, 1.0, 2.0 and 3.0 km s⁻¹) were measured from their time-of-flight between a laser curtain at the end of the launch tube and a piezoelectric impact sensor attached to the target. As a result, velocity measurements were accurate to ±0.01 km s⁻¹. The blast tank was maintained at a vacuum of 0.5 mbar throughout the experiments to prevent slowing of the projectiles due to air resistance and the targets were placed into the blast tank of the LGG at normal incidence to the projectiles' flight path.

A minimum of 25 impact craters on each of the target materials were analyzed after every experiment. A field emission scanning electron microscope (FEG-SEM, Hitachi S-4700) was used to obtain high resolution images of impact craters on the targets. Energy Dispersive X-ray (EDX) microanalysis, using a Bruker Quantax FlatQUAD, was used to identify and detect the abundance of atomic carbon as a tracer for PMMA (C₅O₂H₈), providing a means of assessing particle capture. The image processing software ImageJ (Abrámoff et al. 2004) was used to record the average diameter and area of the craters formed by the different sized particles at given velocities. These data were plotted in order to calculate the particle-crater size calibrations. Micro-Raman spectroscopy, using a LabRam-HR from Horiba incorporating 632 nm excitation laser and 1000× magnification, was used to analyze the organic compounds within the captured material and determine organic survival. The Raman signature of PMMA has a number of well-defined peaks that act as a ‘fingerprint’ that were directly compared with the spectra of captured material to confirm the presence of intact/unmodified PMMA.

### Results

The capture efficiency, organic survival and particle-crater size calibration results are presented in separate sections and the nature of the impacts is described for each particle size and velocity. Illustrative data are shown in Table 2.

Capture Efficiency

The relative capture efficiency of each capture surface (CS) material was quantitatively measured by calculating the mean residue coverage (MRC) per crater across a sample (25+) of craters. This method was selected due to the challenges associated with measuring the volume of residue deposited on the targets, as accurately determining the thickness of the residue (˜50 nm-5 μm) is extremely challenging and destructive. Capture efficiency was primarily affected by the impact velocity and CS material and declined with increasing impact velocities. Capture efficiency varied between the different particle sizes and CS, but maintained a similar trend.

0.5 km s⁻¹ Velocity Impacts

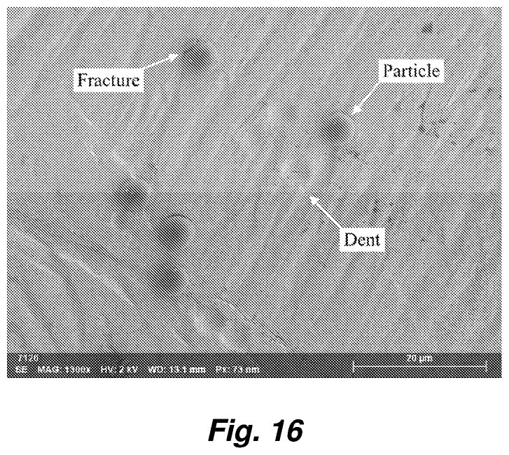

During the 0.5 km s⁻¹ velocity impacts, the particles either stuck to the targets or rebounded, in some cases leaving dents, illustrated in FIG. 16. Typically, very little particle deformation was observed and most of the captured particles maintained their original spherical shape and size, with a small number of particles exhibiting minor fractures, illustrated in FIG. 16.

The sticking coefficient, defined simply as the ratio of stuck-particles-to-dents, was used to calculate the capture efficiency, where the MRC was 100% or 0% for captured whole-particles and dents, respectively. During the very low (0.5 km s⁻¹) velocity impacts it was only possible to directly calculate the sticking coefficient for the indium foil as the harder CS materials did not deform. A uniform impact distribution was assumed across the whole target in order to calculate the sticking coefficient on the remaining CS materials.

The 4 μm diameter particles were captured with the highest efficiency, relative to the other particle sizes, across all of the CS materials at this speed. The highest performing material was indium (66% MRC) and the lowest was aluminum (21% MRC), with an average capture efficiency of 43.4% across all the CS materials. The 6 μm diameter particles were captured with an average MRC of 4% across the CS materials and the 10 μm diameter particles had a similarly low capture efficiency with MRC≤2% across the CS materials. This represents a significant decline in capture compared to the 4 μm particles.

1.0 km s⁻¹ Velocity Impacts

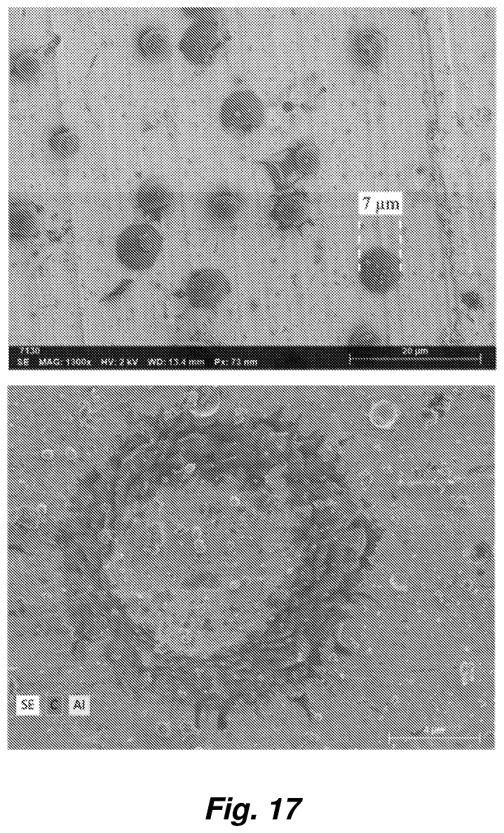

During the 1.0 km s⁻¹ velocity impacts the capture efficiency of the CS materials improved for all sizes of particles. The nature of the impacts was similar to those observed at 0.5 km s⁻¹ velocity, where particles stuck to, or rebounded off, the target. FIG. 17 (left) reveals that, although whole particles were captured on the target, many of the particles exhibited deformation and shape modification. Additionally, rings of carbon-rich residue, presumed to be PMMA, were observed around a large number of dents, demonstrated in FIG. 17 (right). Dents were observed on all of the CS materials and, therefore, direct sticking coefficients were calculated for each material.

The 4 μm diameter particles were captured with an average MRC of 69.2% across the CS materials. The highest performing material was silver (85% MRC), with gold (84%) and indium (82%) exhibiting similarly high capture efficiency. The 6 μm diameter particles were captured with improved efficiency during the 1.0 km s⁻¹ velocity impacts. Indium had the highest MRC (79%) and the lowest was aluminum (3%), with an average capture efficiency of 30.8% across the CS materials. The capture efficiency of the 10 μm diameter particles improved compared to the 0.5 km s⁻¹ velocity impacts, but was still relatively low, with an average MRC of 5.8% with only gold and indium exhibiting significant particle capture.

2.0 km s⁻¹ Velocity Impacts

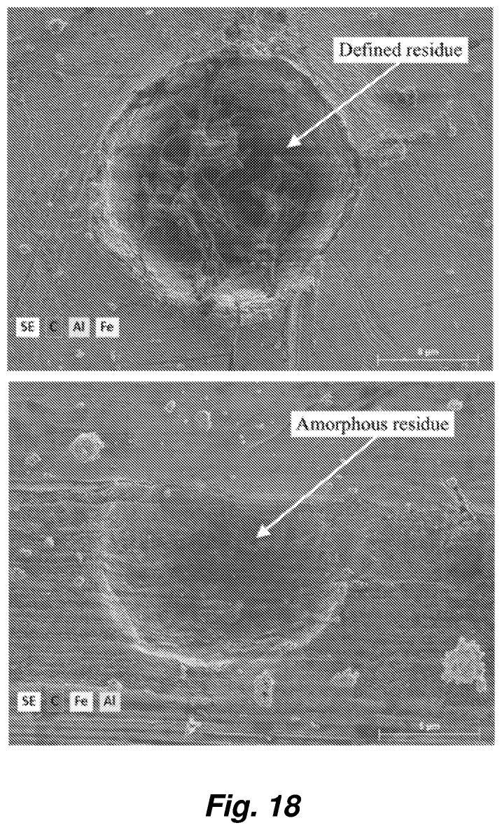

Whole particles were no longer captured by the CS during the 2.0 km s⁻¹ velocity impacts and target deformation increased forming impact craters. Well-defined ‘stringy’ residue—indicative of projectile melting—was observed in certain craters, illustrated in FIG. 18 (left), while others only contained amorphous residue concentrates, FIG. 18 (right). A number of craters exhibited no residue capture. Both types of residue were deposited within the craters, and in some cases, extruded beyond the crater's rim.

The 4 μm diameter particles were captured with an average MRC of 12.4% across the CS materials. The highest performing material was silver (20.4% MRC) and the lowest was copper (3.7% MRC). The 6 μm diameter particles were captured with an average MRC of 12.9% across the CS materials. Indium had the highest capture efficiency (15% MRC) and aluminum had the lowest (7.5% MRC). The capture efficiency was significantly higher for the 10 μm diameter particle impacts, with an average MRC of 25.5%. Similarly, indium had the highest capture efficiency (35.3% MRC) and aluminum had the lowest (14.7% MRC).

3.0 km s⁻¹ Velocity Impacts

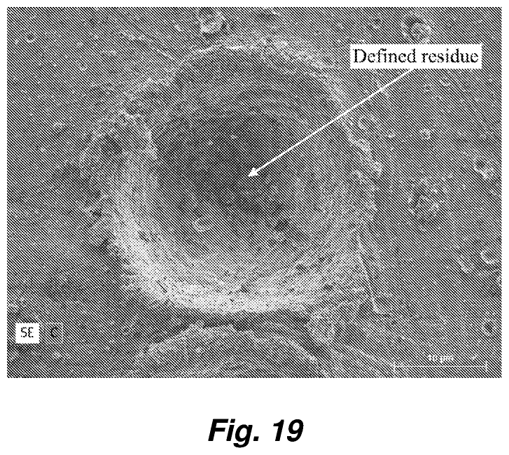

During the 3.0 km s⁻¹ velocity impacts, the capture efficiency across the CS materials significantly decreased for all sizes of particles. The impact nature was similar to those observed at 2.0 km s⁻¹ velocity, where particles were highly disrupted and deposited well-defined stringy residue (FIG. 19) and amorphous concentrates on the CS, but in much lower quantities.

The 4 μm diameter particles were captured with an average MRC of 4.7% across the CS materials. The highest performing material was indium (7.5% MRC) and the lowest was gold (2.0% MRC). The 6 and 10 μm diameter particles had similarly low capture efficiency, with an average MRC of 0.4% and 0.7% respectively. Indium had the highest capture efficiency and was the only CS material for both 6 and 10 μm diameter particles to have MRC>1% at 1.3% and 2.2% respectively.

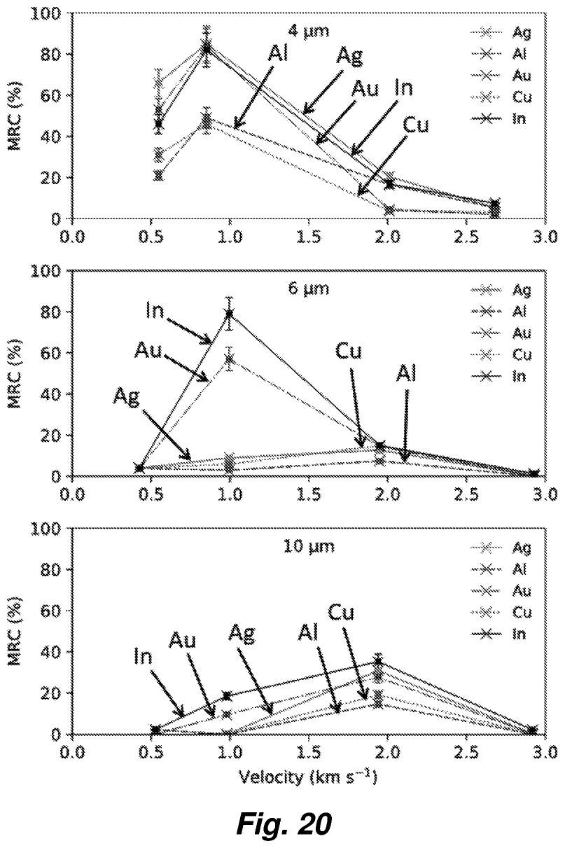

To summarize the capture efficiency data, the MRC was plotted against impact velocity for all the CS materials for each particle diameter (FIG. 20). The plots show that gold, indium and silver represented the best capture mediums across the 0.5-3.0 km s⁻¹ velocity range, with peak capture occurring at ˜1.0 km s⁻¹ for small particles and ˜2.0 km s⁻¹ for larger particles. Data are shown in Table 2.

TABLE 2
Mean residue coverage (MRC) data for the 4, 6 and
10 μm diameter particles on Ag, Al, Au, Cu, and In.

| Particle diameter | Velocity | | | | | |
|---|---|---|---|---|---|---|
| (μm) | (km s⁻¹) | Ag | Al | Au | Cu | In |
| 4 | 0.547 | 66.0 | 21.0 | 53.0 | 31.0 | 46.0 |
| 4 | 0.851 | 85.0 | 49.0 | 84.0 | 46.0 | 82.0 |
| 4 | 2.01 | 20.4 | 16.6 | 4.6 | 3.7 | 16.9 |
| 4 | 2.68 | 5.4 | 5.4 | 2.0 | 3.2 | 7.5 |
| 6 | 0.425 | 4.0 | 4.0 | 4.0 | 4.0 | 4.0 |
| 6 | 0.995 | 9.0 | 3.0 | 57.0 | 6.0 | 79.0 |
| 6 | 1.95 | 12.8 | 7.5 | 14.5 | 14.9 | 15.0 |
| 6 | 2.93 | 0.2 | 0.1 | n.d. | 0.5 | 1.3 |
| 10 | 0.527 | n.d. | 2.3 | n.d. | 2.3 | 2.3 |
| 10 | 0.979 | n.d. | n.d. | 9.6 | n.d. | 18.5 |
| 10 | 1.94 | 30.8 | 14.7 | 27.7 | 19.1 | 35.3 |
| 10 | 2.92 | 0.3 | 0.04 | 0.2 | 0.1 | 2.2 |

Organic Survival

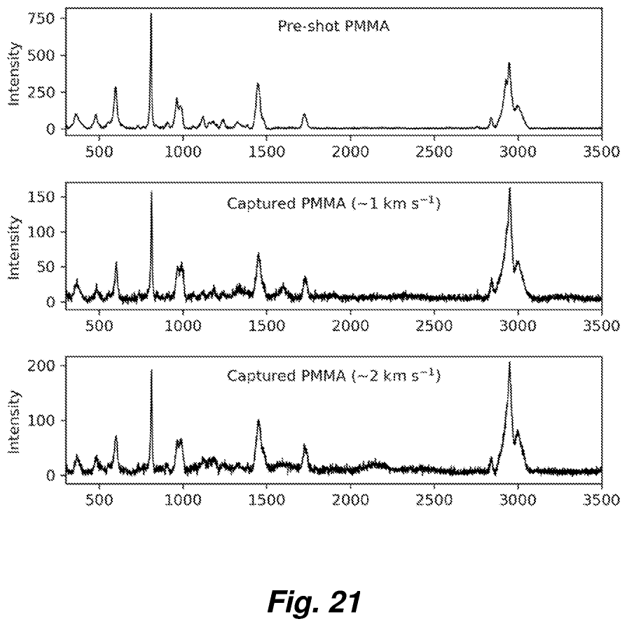

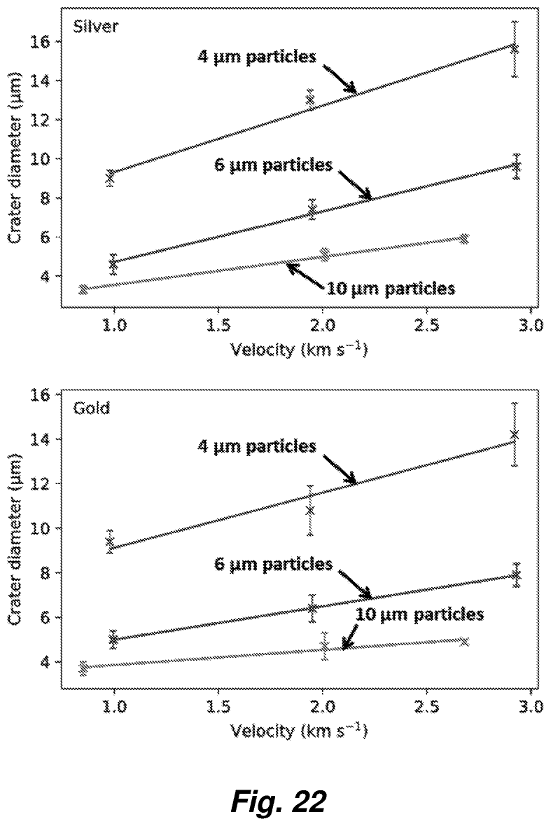

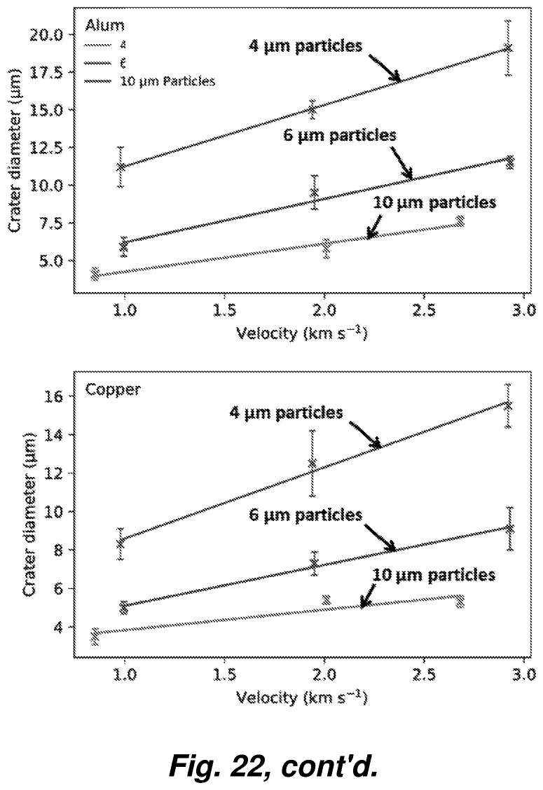

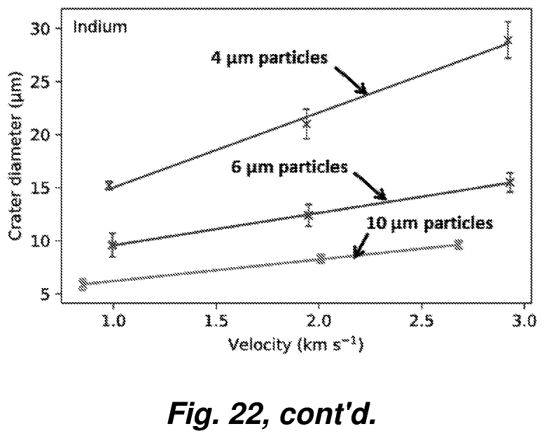

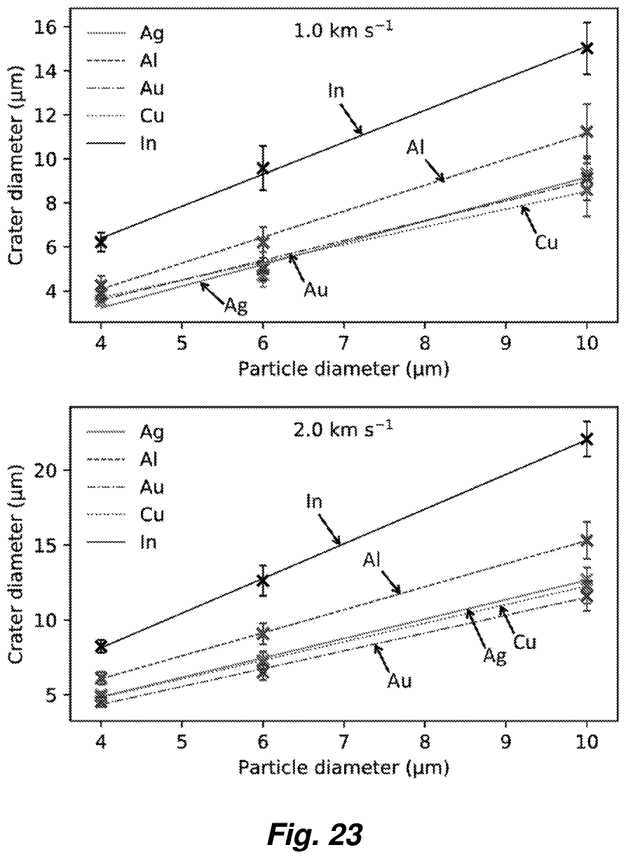

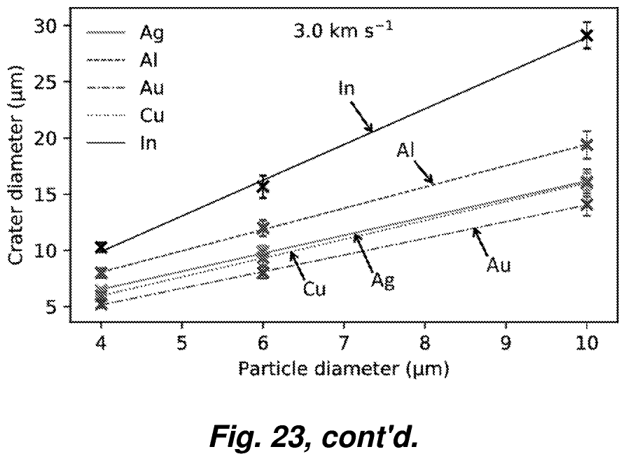

The vibrational spectra of pre-shot PMMA particles were analyzed using a Raman spectrometer to facilitate direct comparison between captured particles and residues (FIG. 21). Peaks in the pre-shot PMMA were observed at 487 cm⁻¹ (C—C in plane bending), 600 cm⁻¹ (C—O in plane bending), 810 cm⁻¹ (C═O in plane bending), 970 cm⁻¹ (C—C stretching), 990 cm⁻¹ (C—C stretching), 1453 cm⁻¹ (CH₃ deformation) and 1730 cm⁻¹ (C═O stretching). These correlate with literature for observed and theoretical Raman wavenumbers for PMMA (Haris et al. 2010).

Raman spectral analysis for the 1.0 and 2.0 km s⁻¹ velocity impacts revealed that the Raman spectra were unchanged between the pre-shot PMMA and the particles and residues captured by the CS—confirming PMMA remains chemically intact and unmodified under these impact conditions. FIG. 21 provides a direct comparison between: (1) pre-shot PMMA particles; (2) a particle captured on the indium target at 0.995 km s⁻¹ velocity; (3) residue captured in a crater on the indium target at 1.94 km s⁻¹ velocity. The low capture efficiency at ˜3.0 km s⁻¹ meant Raman microscopy was inconclusive for residue identification within the craters.

Particle-Crater Size Calibration

The relative size of the craters, with respect to projectile diameter and impact velocity, were measured by averaging the major and minor diameters from the best-fit-ellipse for a sample of 10 craters on each CS material. This method provided a means of calculating the size of circular and irregularly shaped impact craters. The mean crater diameter was plotted against impact velocity for each of the particle diameters and CS materials (FIG. 13). The error bars and shaded regions represent the standard deviation (1σ, n=10) of the crater diameters. A linear correlation between the crater diameter and impact velocity for each particle size and CS material was observed.

The trend lines from the velocity-crater diameter plots can be interpolated at any given velocity to ascertain the particle-crater calibration for each CS material. Particle-crater calibration plots for the velocities studied are provided in FIG. 14. The error bars represent the uncertainty carried forward from the velocity-crater plots and are the mean standard deviation (1σ) across the 1.0-3.0 km s⁻¹ velocity range for each particle size. A linear correlation between the particle- and crater-size is observed. The particle-crater calibration plots will facilitate ice-particle impact experiments by providing a means of calculating the size of an impactor from the crater it creates on impact with a specific target material at a given velocity.

### Discussion

Our overall goal is to explore the feasibility of flying through the ice plumes at Enceladus and gathering sufficient ice particles that we can perform a sensitive analysis for unmodified organic biomarkers using for example the Enceladus Organic Analyzer (Mathies et al. 2017). It is thus important to understand the nature of particle impacts on different target materials, to understand how the impacts change with velocity and particle size, to identify the optimal material for particle capture, and to ascertain the organic survival of ice-particle simulants post impact. We begin here by studying a model ice particle—PMMA—because this polymer has mechanical properties and a phase transition that are similar to ice and it is available in a wide variety of well-defined sizes. These PMMA impacts establish a particle-crater size calibration that will facilitate the interpretation of ice-particle impact experiments in the next phase of this research. Therefore, it was important to select a material with a phase transition at a relevant temperature (160° C. for PMMA) as this not only affects the capture efficiency, but may contribute to the deposition of organic compounds entrained in ice particles.

We performed LGG experiments to study the impacts of PMMA particles into a selection of inert metal target capture surfaces (CS) at different velocities. Organic PMMA ice-particle simulants with diameters of 4, 6 and 10 μm were chosen to imitate the size of particles in Enceladus' plume at an altitude of 50 km. Impact velocities ranging from 0.5-3.0 km s⁻¹ were selected as they approach the likely upper and lower velocity limits of an Enceladus and Saturn orbiter, respectively. Inert metal target foils of Ag, Al, Au, Cu and In were identified as potentially compliant capture materials that meet the science and engineering requirements of the EOA capture system (Mathies et al. 2017). In particular we desire a compliant material that more gradually slows the impacting particle and creates a crater to capture the residue. We also desire a material that can be easily cleaned to provide low background organic levels. Furthermore, the CS must be easily washed to release the captured materials for analysis. Both of these desirements are not easily achieved by low-density porous capture materials such as aerogels. Our results reveal that organic compounds do survive impacts in the velocity range studied and that capture efficiency is influenced by the capture surface, impact velocity and particle size.

At the low velocity 0.5 km s⁻¹ range, whole particles were captured during the impacts with capture efficiency depending on size. SEM-EDX analysis revealed that the captured particles experienced little or no deformation. This suggests that the particles did not melt and fuse to the capture surface on impact, but rather sticking of the particle onto the cratered target was responsible for capture. This hypothesis is supported by the absence of obvious residue in the dents imprinted on the target by rebounded particles. Interestingly, significant capture was only observed for the 4 μm diameter particles, suggesting that the stiction force acting on the heavier 6 and 10 μm particles parallel to the vertically mounted CS was insufficient to keep them bound to the surface. Alternatively, the larger particles with higher kinetic energy rebound off the target with great enough force to overcome the stiction. Gold, indium and silver had the highest capture efficiency of the CS materials for the 0.5 km s⁻¹ velocity impacts. This result is reasonable as the softer materials would yield larger impact craters that would provide greater stiction due to the larger contact area between the CS and the particles. This stiction could be due to Van der Waals interactions between the PMMA and the metals, but it may also be due to partial melting of the PMMA surface, however, there was no evidence of sufficiently thick molten residue in the craters to confirm this hypothesis for the 0.5 km s⁻¹ velocity impacts.

At higher 1.0 km s⁻¹ velocity, the capture efficiency increased for all of the CS materials. This trend could be explained by the increased stiction force between the capture surface and the particles due to deeper surface penetration and enhanced surface contact area. An alternative, or perhaps additional, hypothesis is that these higher velocity impacts have sufficient energy to partially melt the particles causing them to fuse with the target and improve capture. It is therefore expected that CS materials with low thermal conductivity would provide a better capture medium due to increased melting as less thermal energy dissipates from the particles to the target. This hypothesis is supported by the results where indium, the CS with the lowest thermal conductivity (83.7 K), had the highest increase in capture efficiency relative to the other CS materials. Furthermore, SEM-EDX analysis indicated that particles captured on the indium target underwent the highest thermal deformation. However, since impacts into softer indium produce deeper and larger craters we would expect an enhanced stiction process for indium as well. A similar relationship between the particle size and capture efficiency was observed for the 1.0 km s⁻¹ velocity impacts, where the 4 μm diameter particles were captured with highest efficiently, followed by the 6 and 10 μm diameter particles.

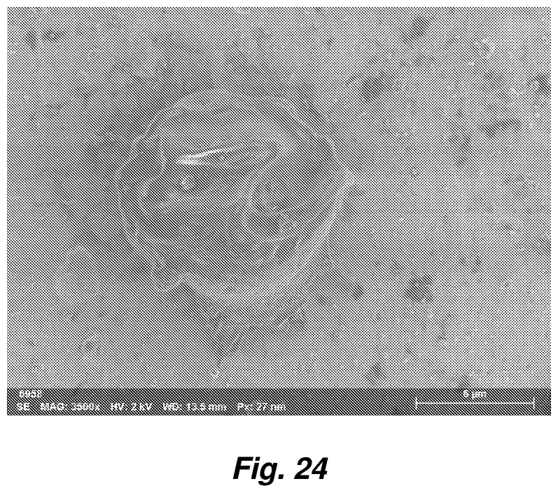

To explore the feasibility of the hypothesis that the particles are melting in the 1.0 km s⁻¹ velocity impacts, we calculated that the energy required to melt a 10 μm diameter PMMA particle starting at room temperature was equal to ˜1.25×10⁻⁷ J. It is generally accepted that during an impact the energy is roughly distributed evenly between the target and the projectile (Gault et al. 1963). Individual PMMA particles with a 10 μm diameter and 1.0 km s⁻¹ velocity have a kinetic energy of ˜2.8×10⁻⁷ J. Assuming 50% of the kinetic energy is transferred to the target, 1.4×10⁻⁷ J of the energy would remain in the projectile, which is 112% of the energy required to heat the whole PMMA particle to melting point (160° C.) from room temperature. This result is supported by SEM analysis that revealed a significant amount of thermal deformation in the particles (FIG. 24) during the 1.0 km s⁻¹ velocity impacts and clear signs of molten residue deposits around the perimeter of numerous craters (FIG. 17).

The nature of the impacts changed considerably for the 2.0 and 3.0 km s⁻¹ velocity experiments. These highly destructive impacts had enough energy to initiate a phase change in the particles, causing substantial thermal disruption. Although residue from the particles was captured inside the craters, projectile mass is lost in the form of PMMA impact ejecta. This resulted in a decrease in capture efficiency across the CS materials, with the interesting exception of the 10 μm diameter particles, that suffered relatively poor capture efficiency for the lower velocity impacts. This suggests that thermal conductivity has little significance for capture efficiency during the highly destructive and energetic impacts, and that pliable materials capable of dissipating kinetic energy through target deformation are better suited to minimize ejecta and increase capture efficiency. This hypothesis is supported by the fact that gold (120 MPa) and indium (4.5 MPa), the two softest CS materials, had the highest capture efficiency for the 2.0 and 3.0 km s⁻¹ velocity impacts.

Micro-Raman spectroscopy was performed on the particles and residues captured during the impacts to determine whether the PMMA organic polymer suffered from significant bond disruption Raman vibrational spectroscopy measures the symmetric vibrations of the polymer and significant bond disruption should alter the observed vibrational frequencies. A direct comparison between the Raman spectra of pre-shot PMMA, captured particles and residue revealed similar spectra. This result confirms that organic compounds remain chemically intact during impacts with velocity ≤2.0 km s⁻¹ Raman microscopy was inconclusive for studying the 3.0 km s⁻¹ velocity impacts due to the low capture efficiency; the residue was possibly too thin and had insufficient cross-section to generate a detectable Raman spectrum. These are important results as they underline the significance of selecting an efficacious encounter velocity for organic sample capture during an Enceladus and potentially Europa fly-by mission.

A linear correlation was established between the impact velocity and crater size for the particles on the different CS materials. Data from these trend lines can be interpolated to particle-crater calibration plots for each CS material at a given impact velocity. Extrapolation beyond the 1.0-3.0 km s⁻¹ velocity range is not advised as craters were not reliably observed below 1.0 km s⁻¹ and above 3.0 km s⁻¹ the particle-crater size relationship may become non-linear due to greater kinetic energy, which increases with the square of the velocity.

### Conclusion

Capturing ice particles during Enceladus plume transits has been identified as a potential method of gathering pristine subsurface ocean samples from Enceladus for in situ chemical analysis. This work shows that capture systems in development can provide successful capture of intact organic ice-simulant particles. These results also reveal how capture efficiency varies with particle size, impact velocity and capture medium.

Any mission designed to collect samples from icy plumes must carefully consider the encounter velocity and capture medium of their collection instrument, if high capture efficiency is desired. Our results indicate optimal capture (˜80% MRC) is achieved for particles with diameters ranging 4-6 μm and an impact velocity of ˜1 km s⁻¹ on indium foil. Under these conditions the particles remain intact, both physically and chemically, and embed in the soft capture medium. Our demonstration that organic particles can be captured in high and hypervelocity impacts on certain capture surfaces without chemical modification is an important step forward.

The particle-crater calibration plots facilitate future ice particle impact experiments necessary for successful development of the EOA capture system. Impact experiments with ice particles entrained with organic compounds are currently being carried out and will provide important knowledge for the development of instruments capable of optimally probing for biosignatures in icy plumes at Enceladus and potentially Europa.

### Acknowledgements

We acknowledge the NASA MATISSE Grant 80NSSC17K0600, Enceladus Organic Analyzer (EOA) and the NASA Instrument Concepts for Europa Exploration 2, ICEE2, Grant 80NSSC19K0616, Microfabricated Organic Analyzer for Biosignatures (MOAB). Matin Golozar thanks the Lin Graduate Fellowship for partial financial support. Richard Mathies thanks the UC Retirement System for financial support. Portions of this work were funded by the Mathies Royalty Fund.

### REFERENCES

Abràmoff, M. D., Magalhaes, P. J. and Ram, S. J., 2004. Image processing with ImageJ. Biophotonics International, 11(7), pp. 36-42.
Avdellidou, C., Price, M. C., Delbo, M., Ioannidis, P. and Cole, M. J., 2015. Survival of the impactor during hypervelocity collisions-I. An analogue for low porosity targets. Monthly Notices of the Royal Astronomical Society, 456(3), pp. 2957-2965.
Beegle, L. and Bhartia, R., 2016, April. SHERLOC: an investigation for Mars 2020. In EGU General Assembly Conference Abstracts (Vol. 18).
Burchell, M. J., Cole, M. J., McDonnell, J. A. M. and Zarnecki, J. C., 1999. Hypervelocity impact studies using the 2 MV Van de Graaff accelerator and two-stage light gas gun of the University of Kent at Canterbury. Measurement Science and Technology, 10(1), p. 41.
Burchell, M. J., Bowden, S. A., Cole, M., Price, M. C. and Parnell, J., 2014. Survival of organic materials in hypervelocity impacts of ice on sand, ice, and water in the laboratory. Astrobiology, 14(6), pp. 473-485.
Brownlee, D. E., Tsou, P., Burnett, D. S., Clark, B., Hanner, M. S., Hörz, F., Kissel, J., McDonnell, J. A. M., Newburn, R. L., Sandford, S. and Sekanina, Z., 1997. The STARDUST mission: returning comet samples to Earth. Meteoritics and Planetary Science, 32(S4), p. A22.
Brownlee, D., Tsou, P., Aléon, J., Alexander, C. M. D., Araki, T., Bajt, S., Baratta, G. A., Bastien, R., Bland, P., Bleuet, P. and Borg, J., 2006. Comet 81P/Wild 2 under a microscope. Science, 314(5806), pp. 1711-1716.
Chiesl, T. N., Chu, W. K., Stockton, A. M., Amashukeli, X., Grunthaner, F. and Mathies, R. A., 2009. Enhanced amine and amino acid analysis using Pacific Blue and the Mars Organic Analyzer microchip capillary electrophoresis system. Analytical chemistry, 81(7), pp. 2537-2544.
Clemett, S. J., Sandford, S. A., Nakamura-Messenger, K., Hoerz, F. and McKay, D. S., 2010. Complex aromatic hydrocarbons in Stardust samples collected from comet 81P/Wild 2. Meteoritics & Planetary Science, 45(5), pp. 701-722.
Elsila, J. E., Glavin, D. P. and Dworkin, J. P., 2009. Cometary glycine detected in samples returned by Stardust. Meteoritics & Planetary Science, 44(9), pp. 1323-1330.
Gault, D. E. and Heitowit, E. D., 1963. The partition of energy for hypervelocity impact craters formed in rock. In Proc. Sixth Hypervelocity Impact Symp. (Vol. 2, pp. 419-456).
Glavin, D. P., Dworkin, J. P. and Sandford, S. A., 2008. Detection of cometary amines in samples returned by Stardust. Meteoritics & Planetary Science, 43(1-2), pp. 399-413.
Goesmann, F., Brinckerhoff, W. B., Raulin, F., Goetz, W., Danell, R. M., Getty, S. A., Siljeström, S., Mißbach, H., Steininger, H., Arevalo Jr, R. D. and Buch, A., 2017. The Mars Organic Molecule Analyzer (MOMA) instrument: characterization of organic material in martian sediments. Astrobiology, 17(6-7), pp. 655-685.
Goldman, N., Reed, E. J., Fried, L. E., Kuo, I. F. W. and Maiti, A., 2010. Synthesis of glycine-containing complexes in impacts of comets on early Earth. Nature Chemistry, 2(11), p. 949.
Haris, M. R. H. M., Kathiresan, S. and Mohan, S., 2010. FT-IR and FT-Raman spectra and normal coordinate analysis of poly methyl methacrylate. Der Pharma Chemica, 2(4), pp. 316-323.
Hedman, M. M., Nicholson, P. D., Showalter, M. R., Brown, R. H., Buratti, B. J. and Clark, R. N., 2009. Spectral observations of the Enceladus plume with Cassini-VIMS. The Astrophysical Journal, 693(2), p. 1749.
Hibbert, R., Cole, M. J., Price, M. C. and Burchell, M. J., 2017. The Hypervelocity Impact Facility at the University of Kent: Recent Upgrades and Specialized Capabilities. Procedia Engineering, 204, pp. 208-214.
Hsu, H. W., Postberg, F., Sekine, Y., Shibuya, T., Kempf, S., Horányi, M., Juhász, A., Altobelli, N., Suzuki, K., Masaki, Y. and Kuwatani, T., 2015. Ongoing hydrothermal activities within Enceladus. Nature, 519(7542), p. 207.
Kearsley, A. T., Burchell, M. J., Price, M. C., Green, S. F., Franchi, I. A., Bridges, J. C., Starkey, N. and Cole, M. C., 2010. Distinctive impact craters are formed by organic-rich cometary dust grains (abstract #1435). In 41st Lunar and Planetary Science Conference. CD-ROM.
Kelley, D. S., Karson, J. A., Früh-Green, G. L., Yoerger, D. R., Shank, T. M., Butterfield, D. A., Hayes, J. M., Schrenk, M. O., Olson, E. J., Proskurowski, G. and Jakuba, M., 2005. A serpentinite-hosted ecosystem: the Lost City hydrothermal field. Science, 307(5714), pp. 1428-1434.
Leitner, J., Stephan, T., Kearsley, A. T., Hörz, F., Flynn, G. J. and Sandford, S. A., 2008. TOF-SIMS analysis of crater residues from Wild 2 cometary particles on Stardust aluminum foil. Meteoritics & Planetary Science, 43(1-2), pp. 161-185.
Mahaffy, P. R., Webster, C. R., Cabane, M., Conrad, P. G., Coll, P., Atreya, S. K., Arvey, R., Barciniak, M., Benna, M., Bleacher, L. and Brinckerhoff, W. B., 2012. The sample analysis at Mars investigation and instrument suite. Space Science Reviews, 170(1-4), pp. 401-478.
Mathies, R. A., Razu, M. E., Kim, J., Stockton, A. M., Turin, P. and Butterworth, A., 2017. Feasibility of detecting bioorganic compounds in Enceladus plumes with the Enceladus Organic Analyzer. Astrobiology, 17(9), pp. 902-912.
Melosh, H. J., 1989. Impact cratering: A geologic process. Research supported by NASA. New York, Oxford University Press (Oxford Monographs on Geology and Geophysics, No. 11), 1989, 253 p., 11.
Parnell, J., Bowden, S., Lindgren, P., Burchell, M., Milner, D., Price, M., Baldwin, E. C. and Crawford, I. A., 2010. The preservation of fossil biomarkers during meteorite impact events: Experimental evidence from biomarker-rich projectiles and target rocks. Meteoritics & Planetary Science, 45(8), pp. 1340-1358.
Porco, C., DiNino, D. and Nimmo, F., 2014. How the geysers, tidal stresses, and thermal emission across the south polar terrain of Enceladus are related. The Astronomical Journal, 148(3), p. 45.
Postberg, F., Kempf, S., Schmidt, J., Brilliantov, N., Beinsen, A., Abel, B., Buck, U. and Srama, R., 2009. Sodium salts in E-ring ice grains from an ocean below the surface of Enceladus. Nature, 459(7250), p. 1098.
Postberg, F., Schmidt, J., Hillier, J., Kempf, S. and Srama, R., 2011. A salt-water reservoir as the source of a compositionally stratified plume on Enceladus. Nature, 474(7353), p. 620.
Postberg, F., Khawaja, N., Abel, B., Choblet, G., Glein, C. R., Gudipati, M. S., Henderson, B. L., Hsu, H. W., Kempf, S., Klenner, F. and Moragas-Klostermeyer, G., 2018. Macromolecular organic compounds from the depths of Enceladus. Nature, 558(7711), p. 564.
Price, M. C., Burchell, M., Kearsley, A. T. and Cole, M. J., 2012, March. Alteration and formation of organic molecules via hypervelocity impacts. In Lunar and Planetary Science Conference (Vol. 43).
Schneider, N. M., Burger, M. H., Schaller, E. L., Brown, M. E., Johnson, R. E., Kargel, J. S., Dougherty, M. K. and Achilleos, N. A., 2009. No sodium in the vapour plumes of Enceladus. Nature, 459(7250), p. 1102.
Skelley, A. M., Scherer, J. R., Aubrey, A. D., Grover, W. H., Ivester, R. H., Ehrenfreund, P., Grunthaner, F. J., Bada, J. L. and Mathies, R. A., 2005. Development and evaluation of a microdevice for amino acid biomarker detection and analysis on Mars. Proceedings of the National Academy of Sciences, USA 102(4), pp. 1041-1046.
Skelley, A. M., Aubrey, A. D., Willis, P. A., Amashukeli, X., Ehrenfreund, P., Bada, J. L., Grunthaner, F. J. and Mathies, R. A., 2007. Organic amine biomarker detection in the Yungay region of the Atacama Desert with the Urey instrument. Journal of Geophysical Research: Biogeosciences, 112(G4).
Spahn, F., Schmidt, J., Albers, N., Horning, M., Makuch, M., Seiβ, M., Kempf, S., Srama, R., Dikarev, V., Helfert, S. and Moragas-Klostermeyer, G., 2006. Cassini dust measurements at Enceladus and implications for the origin of the E ring. Science, 311(5766), pp. 1416-1418.
Waite, J. H., Combi, M. R., Ip, W. H., Cravens, T. E., McNutt, R. L., Kasprzak, W., Yelle, R., Luhmann, J., Niemann, H., Gell, D. and Magee, B., 2006. Cassini ion and neutral mass spectrometer: Enceladus plume composition and structure. Science, 311(5766), pp. 1419-1422.
Waite Jr, J. H., Lewis, W. S., Magee, B. A., Lunine, J. I., McKinnon, W. B., Glein, C. R., Mousis, O., Young, D. T., Brockwell, T., Westlake, J. and Nguyen, M. J., 2009. Liquid water on Enceladus from observations of ammonia and 40 Ar in the plume. Nature, 460(7254), p. 487.
Wickham-Eade, J. E., Burchell, M. J., Price, M. C. and Harriss, K. H., 2018. Hypervelocity impact fragmentation of basalt and shale projectiles. Icarus, 311, pp. 52-68.
Zolotov, M. Y., 2007. An oceanic composition on early and today's Enceladus. Geophysical Research Letters, 34(23).

### Example 2

### Ice Particle Impact Experiments

Frozen ice sabots were made using a Pacific Blue fluorescent dye and cysteine-water solution (12.5:4000 μL). The sabots were then accelerated to 0.5-3.0 km s⁻¹ before impacting targets of Al, Au, Cu and In. Silver was dropped as it had similar capture properties to Gold. As described below fluorescent microscopy of the Pacific Blue residue provides quantitative capture analysis. SEM/EDX imaging of the cysteine residue provides qualitative capture analysis.

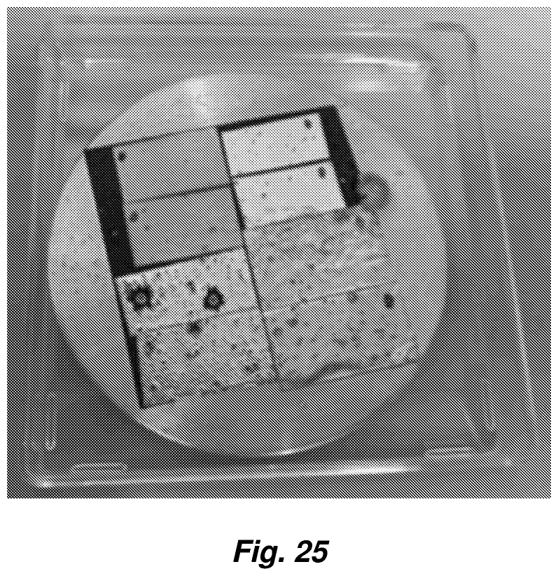

A total of 5 ice shots were conducted. The first 4 shots were used to refine the ice shot protocol. The fifth shot yielded good results with craters on all of the foils which are now in the process of analysis (FIG. 25).

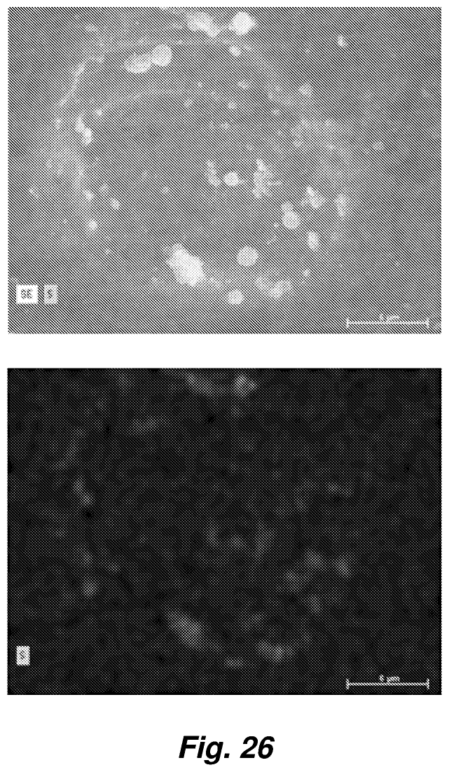

Analysis of Aluminum and Indium foils confirm that cysteine residue is captured within craters. Sulphur was used as a tracer and can be seen within the crater in FIG. 26 (Aluminum). Indium performed similarly.

### Ice Particle Organic Capture Quantitation

To evaluate both the capture and molecular survival of organic biomolecules in high velocity ice impacts we are using a fluorescent dye, Pacific Blue, doped into the ice solution. The idea is to then examine the targets and measure the amount of PB that is captured and survives by epifluorescence microscopy. Since amino acids are likely more stable to shock and heating than an organic dye molecule, this provides as conservative estimate of capture and survival. To this end a fluorescence microscope was set up to interrogate the targets with the appropriate excitation and filters for PB. Initial experiments indicated that the dye in the dry impact residue was relatively non-fluorescent when present as a dry film but exposing the surface to a controlled 70-80% humidity nicely restored its strong fluorescence. This response to humidity also enabled us to verify that the observed fluorescence was indeed due to PB as opposed to other possible fluorescent contaminants deposited in the light gas gun experiment.

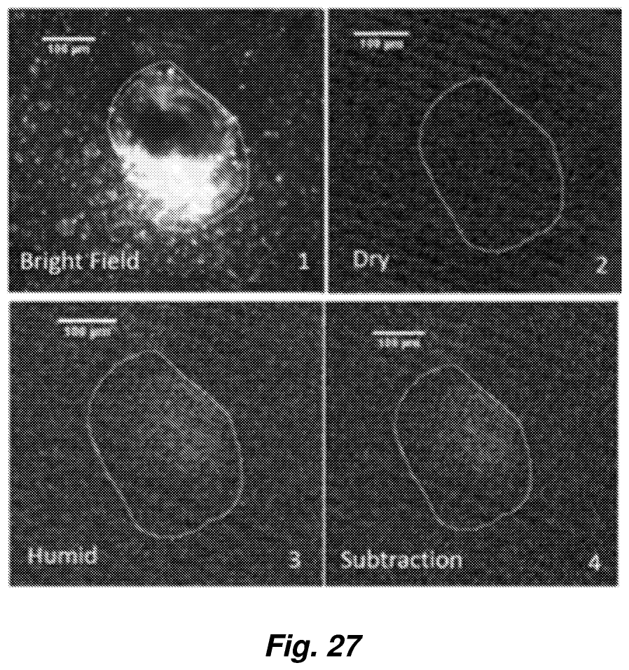

Initial experiments to develop and verify the analytical procedure were performed examining large 100 micron craters at low 4× magnification (Ice shot G0105191). Since this experiment did not include salt in the solution, the ice projectile does not readily break up on acceleration so the particle distribution at the target is large compared to later experiments with salt included. FIG. 27 presents images of a ˜200 micron diameter crater in gold showing the bright field image, an image of the same dry crater, an image of the crater exhibiting fluorescence after hydration as well as the difference image quantitating the captured organic material on the target.

Since this shot was performed at 1.7 km/s this demonstrates significant organic molecule capture and survival at a relatively high impact velocity. This image also establishes the basic feasibility of our approach.

The calibration method for quantitation of PB in the microscope is undergoing revision to improve both precision and accuracy of this measurement. Currently work is focused on looking at smaller craters of more relevance to Enceladus impacts with a 10× and 20× objectives and improving the accuracy of our PB quantitation in the craters. While there is much to do, but we are very excited that the ice shots are working and that we have developed a successful method for quantitating organic capture and survival for hypervelocity target impacts.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A particle capture surface configured for capture of high and/or hyper velocity dust, aerosol, and/or ice particles, wherein said capture surface is comprised of soft metal that maximizes particle capture efficiency, minimizes thermal degradation and shock degradation of chemical and biochemical components in the particles, and said surface is configured to present the captured particles, or components therein, on said surface for direct analysis or to deliver said particles, or component therein, to an analyzer for chemical and/or biochemical analysis of the particles and their component contents.

2. The particle capture surface of claim 1, wherein said surface is configured to deliver said particles to an analyzer for chemical and/or biochemical analysis of the particles and their contents.

3. The particle capture surface according to any one of claims 1-2, wherein said surface is configured to capture extraterrestrial dust, aerosol, and/or ice particles.

4. The particle capture surface according to any one of claims 1-2, wherein said surface is configured to capture extraterrestrial dust, aerosol, and/or ice particles in high earth orbit.

5. The particle capture surface according to any one of claims 1-2, wherein said surface is configured to capture extraterrestrial dust, aerosol, and/or ice particles at high altitude.

6. The particle capture surface according to any one of claims 1-5, wherein said capture surface is configured to provide a particle capture efficiency of at least 0.01%, or at least 0.1%, or at least 0.5%, or at least 1%, or at least 10%, or at least 30%, or at least 50%, or at least 80%, or at least 90% for particles, or at least 95%, or at least 98% up to 100%.

7. The particle capture surface of claim 6, wherein said capture surface is configured to provide a particle capture efficiency ranging from about 1% up to about 50%.

8. The particle capture surface according to any one of claims 6-7, wherein said capture efficiency is for particles impacting said capture surface at an angle ranging from about 45 degrees to about 90 degrees.

9. The particle capture surface of claim 8, wherein said capture efficiency is for particles impacting said capture surface at an angle of about 90 degrees.

10. The particle capture surface according to any one of claims 1-9, wherein said surface is configured to perform said capturing at an average relative velocity of said capture surface and dust and ice particles ranging from about 1 m/s, or from about 10 m/s, or from about 100 m/s, or from about 500 m/s, or from about 1 km/s, up to about 10 km/s, or up to about 5 km/s, or up to about 2.5 km/s, or up to about 1 km/s.

11. The particle capture surface of claim 10, wherein said surface is configured to perform said capturing at an average relative velocity of said capture surface and dust and ice particles ranging from about 1 m/s up to about 5 km/s, or from about 100 m/s up to about 5 km/s, or from about 500 m/s up to about 1 km/s up to about 5 km/s.

12. The particle capture surface according to any one of claims 1-11, wherein said thermal degradation and shock degradation is sufficiently low to permit dispositive identification of at least about 5%, or at least about 10%, or at least about 20%, or at least about 30%, or at least about 40%, or at least about 50%, or at least about 60%, or at least about 70%, or at least about 80%, or at least about 90%, or at least about 95%, or at least about 98% of the organic compounds captured on said surface.

13. The particle capture surface of claim 12, wherein said dispositive identification is by Raman spectroscopy.

14. The particle capture surface of claim 12, wherein said dispositive identification is by optical absorption or emission microscopy or SEM.

15. The particle capture surface of claim 12, wherein said dispositive identification is by a programmable microfluidic analyzer (PMA).

16. The particle capture surface of claim 12, wherein said dispositive identification is by a mass spectroscopy (e.g., laser desorption mass spectroscopy).

17. The particle capture surface according to any one of claims 1-16, wherein said surface is configured to capture particles impacting said surface an angle between about 45 degrees and about 90 degrees.

18. The particle capture surface according to any one of claims 1-17, wherein the average size of said aerosol, ice or dust particles ranges from about 0.1 μm, or from about 1 μm, or from about 2 μm up to about 1000 μm, or up to about 500 μm, or up to about 100 μm, or up to about 50 μm, or up to about 20 μm in diameter.

19. The particle capture surface of claim 18, wherein the average size of said aerosol, ice or dust particles ranges from about 0.1 μm up to about 20 μm.

20. The particle capture surface according to any one of claims 1-19, wherein the projected area of said capture surface area ranges from about 1 cm², or from about 5 cm², or from about 10 cm², or from about 20 cm², or from about 30 cm², or from about 40 cm², or about 50 cm², or from about 60 cm², or from about 70 cm², or from about 80 cm², or from about 90 cm², or from about 100 cm², up to about 1,000 cm², or up to about 500 cm², or up to about 400 cm², or up to about 300 cm², or up to about 200 cm², or up about 190 cm², or up to about 180 cm², or up to about 170 cm², or up to about 160 cm², or up to about 150 cm².

21. The particle capture surface of claim 20, wherein the projected area of said capture surface ranges from about 10 cm² up to about 200 cm², or from about 20 cm² up to about 150 cm², or from about 50 cm² up to about 120 cm².

22. The particle capture surface according to any one of claims 1-21, wherein the shape of the projected area of said capture surface comprise a shape selected from the group consisting of circular, triangular, square, rectangular, hexagonal, and the like.

23. The particle capture surface of claim 22, wherein the shape of the projected area of said capture surface is circular.

24. The particle capture surface of claim 23, wherein the projected area of said capture surface has a diameter of about 10 cm.

25. The particle capture surface according to any one of claims 1-24, wherein said soft capture surface is comprised of a metal selected from the group consisting of Al, Au, Ag, Cu, mercury, gallium, indium, lead, brass, and bronze, or any other soft metal or alloy with similar mechanical properties.

26. The particle capture surface according to any one of claims 1-25, wherein said capture surface is comprised of one, or two or more different soft metal layers where the metals and their thicknesses simultaneously provide both efficient capture and minimal degradation of the chemicals in the particles.

27. The particle capture surface of claim 26, wherein one or more of said layers ranges in thickness from about a few microns up to about a few mm.

28. The particle capture surface of claim 26, wherein one or more of said layers ranges in thickness from about 1 μm, or from about 2 μm, or from about 5 μm, or from about 10 μm, or from about 20 μm, or from about 50 μm, or from about 100 μm, or from about 500 μm up to about 10 mm, or up to about 5 mm, or up to about 4 mm, or up to about 3 mm, or up to about 2 mm, or up to about 1 mm.

29. The particle capture surface according to any one of claims 1-28, wherein said particle capture surface comprises a soft metal disposed on top of a harder metal or a silica substrate.

30. The particle capture surface of claim 29, wherein said particle capture surface comprises a soft metal disposed on top of a harder metal or other material.

31. The particle capture surface of claim 30, wherein said particle capture surface comprises a gold layer disposed on an aluminum and/or silver layer.

32. The particle capture surface of claim 31, wherein said particle capture surface comprises a gold layer disposed on an aluminum layer.

33. The particle capture surface according to any one of claims 1-32 wherein said capture surface is configured to present captured particles for chemical and biochemical assay by optical spectroscopy, optical microscopy, SEM, or mass spectrometry.

34. The particle capture surface according to any one of claims 1-33, wherein said capture surface is configured to present captured particles for chemical and biochemical assay by Raman spectroscopy or Raman microscopy.

35. The particle capture surface according to any one of claims 1-34, wherein said capture surface comprises 2 or more, or 3 or more, or 4 or more or 5 or more different regions comprising different materials and/or material thicknesses to produce different hardnesses.

36. The particle capture surface of claim 35, wherein said capture surface comprises 2 or more, or 3 or more, or 4 or more or 5 or more different regions comprising different materials and/or material thicknesses to simultaneously provide optimal capture of particles having different velocities.

37. The particle capture surface according to any one of claims 1-34, wherein metals comprising said capture surface vary in thickness and/or composition to provide a gradient in hardness across said surface.

38. The particle capture surface according to any one of claims 1-37, wherein said capture surface comprises a component in an aircraft, rocket, satellite or space probe.

39. A particle capture surface configured for capture of high and/or hyper velocity dust, aerosol, and/or ice particles, wherein said capture surface is comprised of an easily cleaned soft metal that maximizes particle capture efficiency, minimizes thermal degradation of chemicals and biochemicals in the particles, that is configured to permit facile dissolution of the particles and their chemical and biochemical contents into a volume of extractant fluid, and is configured to enable transfer of the extractant fluid to an analyzer for chemical and biochemical analysis.

40. The particle capture surface of claim 39, wherein said surface is configured to capture extraterrestrial dust, aerosol, and/or ice particles.

41. The particle capture surface of claim 39, wherein said surface is configured to capture extraterrestrial dust, aerosol, and/or ice particles in high earth orbit.

42. The particle capture surface of claim 39, wherein said surface is configured to capture extraterrestrial dust, aerosol, and/or ice particles at high altitude.

43. The particle capture surface according to any one of claims 39-42, wherein said capture surface is configured to provide a surface in an open chamber configured to pass fluid across said surface to surface to dissolve chemical/biochemical contents of said particles.

44. The particle capture surface according to any one of claims 39-42, wherein said capture surface comprises features that, when said surface is capped with a lid, said features provide one or more channels that direct the flow of a fluid over said surface to dissolve chemical/biochemical contents of said particles.

45. The particle capture surface of claim 44, wherein said one or more channels comprise a serpentine channel that directs flow from an inlet port to an outlet port.

46. The particle capture surface of claim 45, wherein said one or more channels comprise a spiral channel pattern that directs flow from an inlet port to an outlet port.

47. The particle capture surface of claim 46, wherein said one or more channels comprise a square spiral serpentine channel.

48. The particle capture surface of claim 46, wherein said one or more channels comprise a circular spiral serpentine channel.

49. The particle capture surface of claim 46, wherein said one or more channels comprise a switchback serpentine channel.

50. The particle capture surface of claim 44, wherein said one or more channels comprise a branched channel pattern that directs flow from an inlet port to an outlet port.

51. The particle capture surface according to any of claims 44-50, wherein said one or more channels range in depth from about 10 μm, or from about 20 μm, or from about 30 μm, or from about 40 μm up to about 300 μm, or up to about 200 μm, or up to about 100 μm, or up to about 70 μm, or up to about 60 μm, or up to about 50 μm, or up to about 30 μm.

52. The particle capture surface according to any of claims 44-51, wherein said one or more channels range in width from about 20 μm, or from about 30 μm, or from about 40 μm, or from about 50 μm up to about 1000 μm, or to about 500 μm, or up to about 200 μm, or up to about 100 μm.

53. The particle capture surface according to any of claims 44-51, wherein said one or more channels have a depth of about 100 μm and a width of about 400 μm.

54. The particle capture surface according to any one of claims 52-53, wherein said channels have a spacing (between channels) of about 125 μm.

55. The particle capture surface according to any of claims 44-54, wherein said one or more channels have a square or rectangular cross-section, a cross-section with chamfered sides, a cross-section with a curved bottom, and a cross-section with sloping sides, or a conical cross-section.

56. The particle capture surface of claim 64, wherein said one or more channels have a cross-section that is not square or rectangular.

57. The particle capture surface of claim 64, wherein said one or more channels have a cross-section with chamfered sides, a cross-section with a curved bottom, and a cross-section with sloping sides, or a conical cross-section.

58. The particle capture surface according to any of claims 44-57, wherein said features comprise a compliant top coat to improve sealing to a juxtaposed surface.

59. The particle capture surface of claim 58, wherein said compliant top coat comprises a gasket material.

60. The particle capture surface of claim 59, wherein said compliant top coat comprises a soft metal gasket material.

61. The particle capture surface of claim 58, wherein said soft metal gasket material comprises indium.

62. The particle capture surface according to any of claims 44-61, wherein, wherein said features comprise a hydrophobic barrier that prevents wetting in a thin gap between the features and a juxtaposed surface.

63. The particle capture surface of claim 61, wherein said hydrophobic barrier is comprised of a gold overcoat with a hydrophobic thiol coating.

64. The particle capture surface according to any one of claims 39-63, wherein said capture surface is configured to provide a particle capture efficiency of at least 0.01%, or at least 0.1%, or at least 0.5%, or at least 1% cm², or at least 10% cm², or at least 30% cm², or at least 50% cm², or at least 80% cm², of at least 90% for particles cm², or at least 95% cm², or at least 98%.

65. The particle capture surface of claim 64, wherein said capture surface is configured to provide a particle capture efficiency ranging from about 1% up to about 50%.

66. The particle capture surface according to any one of claims 64-65, wherein said capture efficiency is for particles impacting said capture surface at an angle ranging from about 45 degrees to about 90 degrees.

67. The particle capture surface of claim 66, wherein said capture efficiency is for particles impacting said capture surface at an angle of about 90 degrees.

68. The particle capture surface according to any one of claims 64-67, wherein said surface is configured to perform said capturing at an average relative velocity of said capture surface and dust and ice particles ranging from about 1 m/s, or from about 10 m/s, or from about 100 m/s, or from about 500 m/s, or from about 1 km/s, up to about 10 km/s, or up to about 5 km/s, or up to about 2.5 km/s, or up to about 1 km/s.

69. The particle capture surface of claim 68, wherein said surface is configured to perform said capturing at an average relative velocity of said capture surface and dust and ice particles ranging from about 1 m/s up to about 5 km/s, or from about 100 m/s up to about 5 km/s, or from about 500 m/s up to about 1 km/s up to about 5 km/s.

70. The particle capture surface according to any one of claims 64-69, wherein said thermal degradation is sufficiently low to permit dispositive identification of at least about 5%, or at least about 10%, or at least about 20%, or at least about 30%, or at least about 40%, or at least about 50%, or at least about 60%, or at least about 70%, or at least about 80%, or at least about 90%, or at least about 95%, or at least about 98% of the organic compounds captured on said surface.

71. The particle capture surface of claim 70, wherein said dispositive identification is by Raman spectroscopy.

72. The particle capture surface of claim 70, wherein said dispositive identification is by optical microscopy or SEM.

73. The particle capture surface of claim 70, wherein said dispositive identification is by mass spectroscopy (e.g., laser desorption mass spectroscopy).

74. The particle capture surface of claim 70, wherein said dispositive identification is by a programmable microfluidic analyzer (PMA).

75. The particle capture surface of claim 70, wherein said dispositive identification is by a mass spectroscopy (e.g., laser desorption mass spectroscopy).

76. The particle capture surface according to any one of claims 64-75, wherein the average size of said aerosol, ice or dust particles ranges from about 0.1 μm, or from about 1 μm, or from about 2 μm up to about 1000 μm, or up to about 500 μm, or up to about 100 μm, or up to about 50 μm, or up to about 20 μm in diameter.

77. The particle capture surface of claim 76, wherein the average size of said aerosol, ice or dust particles ranges from about 0.1 μm up to about 20 μm.

78. The particle capture surface according to any one of claims 39-77, wherein said surface is configured to capture particles impacting said surface an angle between about 45 degrees and about 90 degrees.

79. The particle capture surface according to any one of claims 64-78, wherein the projected area of said capture surface area ranges from about 1 cm², or from about 5 cm², or from about 10 cm², or from about 20 cm², or from about 30 cm², or from about 40 cm², or about 50 cm², or from about 60 cm², or from about 70 cm², or from about 80 cm², or from about 90 cm², or from about 100 cm², up to about 1000 cm², or up to about 500 cm², or up to about 400 cm², or up to about 300 cm², or up to about 200 cm², or up about 190 cm², or up to about 180 cm², or up to about 170 cm², or up to about 160 cm², or up to about 150 cm².

80. The particle capture surface of claim 79, wherein the projected area of said capture surface ranges from about 10 cm² up to about 200 cm², or from about 20 cm² up to about 150 cm², or from about 50 cm² up to about 120 cm².

81. The particle capture surface according to any one of claims 39-80, wherein the shape of the projected area of said capture surface comprises a shape selected from the group consisting of circular, triangular, square, rectangular, and hexagonal.

82. The particle capture surface of claim 81, wherein the shape of the projected area of said capture surface is circular.

83. The particle capture surface of claim 82, wherein the projected area of said capture surface has a diameter of about 10 cm.

84. The particle capture surface according to any one of claims 39-83, wherein said soft capture surface is comprised of a metal selected from the group consisting of Al, Au, Ag, Cu, mercury, gallium, indium, lead, brass, and bronze, or any other soft metal or alloy with similar mechanical properties.

85. The particle capture surface according to any one of claims 39-84, wherein said capture surface is comprised of one, or two or more different soft metal layers where the metals and their thicknesses simultaneously provide both efficient capture and minimal degradation of the chemicals in the particles.

86. The particle capture surface of claim 85, wherein one or more of said layers ranges in thickness from about a few microns up to about a few mm.

87. The particle capture surface of claim 85, wherein one or more of said layers ranges in thickness from about 1 μm, or from about 2 μm, or from about 5 μm, or from about 10 μm, or from about 20 μm, or from about 50 μm, or from about 100 μm, or from about 500 μm up to about 10 mm, or up to about 5 mm, or up to about 4 mm, or up to about 3 mm, or up to about 2 mm, or up to about 1 mm.

88. The particle capture surface according to any one of claims 39-87, wherein said particle capture surface comprises a soft metal disposed on top of a harder metal or a silica substrate.

89. The particle capture surface of claim 88, wherein said particle capture surface comprises a soft metal disposed on top of a harder metal.

90. The particle capture surface of claim 89, wherein said particle capture surface comprises a gold layer disposed on an aluminum and/or silver layer.

91. The particle capture surface of claim 90, wherein said particle capture surface comprises a gold layer disposed on an aluminum layer.

92. The particle capture surface of claim 88, wherein said particle capture surface comprise a gold layer disposed on an aluminum and/or silver layer.

93. The particle capture surface according to any one of claims 39-92, wherein said capture surface comprises 2 or more, or 3 or more, or 4 or more or 5 or more different regions comprising different materials and/or material thicknesses to produce different hardnesses.

94. The particle capture surface of claim 93, wherein said capture surface comprises 2 or more, or 3 or more, or 4 or more or 5 or more different regions comprising different materials and/or material thicknesses to simultaneously provide optimal capture of particles having different velocities.

95. The particle capture surface according to any one of claims 39-92, wherein metals comprising said capture surface various in thickness and/or composition to provide a gradient in hardness across said surface.

96. The particle capture surface according to any one of claims 39-95, wherein said capture surface comprises a component in an aircraft, rocket, satellite or space probe.

97. A particle capture chamber for capture of high velocity dust and ice particles, said chamber comprising:
a first particle capture surface according to any one of claims 1-37; and
a moveable lid where said lid is configured so that when said capture chamber is closed said lid covers said particle capture surface and with said capture surface forms a sample chamber.

98. The particle capture chamber of claim 97, wherein said surface is configured to capture extraterrestrial dust, aerosol, and/or ice particles.

99. The particle capture chamber of claim 97, wherein said surface is configured to capture extraterrestrial dust, aerosol, and/or ice particles in high earth orbit.

100. The particle capture chamber of claim 97, wherein said surface is configured to capture extraterrestrial dust, aerosol, and/or ice particles at high altitude.

101. The particle capture chamber according to any one of claims 97-100, wherein said lid is configured to slide open.

102. The particle capture chamber according to any one of claims 97-100, wherein said lid is hinged such that it can open providing enhanced material capture by permitting particle capture on said first particle capture surface and on a second particle capture surface disposed on said lid, wherein said second particle capture surface also comprises a particle capture surface according to any one of claims 1-37.

103. The particle capture chamber of claim 102, wherein said first particle capture surface and said second particle capture surface are the same materials and configuration.

104. The particle capture chamber of claim 102, wherein said first particle capture surface and said second particle capture surface are the different materials and/or configuration.

105. The particle capture chamber according to any one of claims 97-104, wherein said sample chamber is configured with an inlet and outlet port and is configured to wash said first capture surface and, when present said second capture surface, and deliver dust and ice particles and their contents to a programmable microfluidic analyzer (PMA) operably coupled to said capture chamber.

106. The particle capture chamber of claim 105, wherein said PMA comprises:
a plurality of pneumatic inputs;
a plurality of microfluidic channels; and
a plurality of μCE separation channels, where said pneumatic inputs microfluidic channels and μCE separation channels are configured to so that fluid samples enter and leave the processor through access ports; wherein
an array of valves drive fluid routing on the PMA; and
sample and reagent storage is provided in addressable wells at the top.

107. The particle capture chamber of claim 106, wherein said PMA permits analysis of different samples.

108. The particle capture chamber according to any one of claims 97-107, wherein said capture surface comprises a component in an aircraft, a rocket, a satellite or space probe.

109. A particle capture chamber for capture of high velocity dust and ice particles, said chamber comprising:
a first particle capture surface according to any one of claims 39-92; and
a moveable lid where said lid is configured so that when said capture chamber is closed said lid covers said particle capture surface and with said capture surface forms a sample chamber that permits facile dissolution of the particles and their chemical and biochemical contents into a volume of extractant fluid and that enables transfer of the extractant fluid to an analyzer for chemical/biochemical analysis.

110. The particle capture chamber of claim 109, wherein said surface is configured to capture extraterrestrial dust, aerosol, and/or ice particles.

111. The particle capture chamber of claim 109, wherein said surface is configured to capture extraterrestrial dust, aerosol, and/or ice particles in high earth orbit.

112. The particle capture chamber of claim 109, wherein said surface is configured to capture extraterrestrial dust, aerosol, and/or ice particles at high altitude.

113. The particle capture chamber according to any one of claims 109-112, wherein said lid is configured to slide open.

114. The particle capture chamber according to any one of claims 109-112, wherein said lid is hinged such that it can open providing enhanced material capture by permitting particle capture on said first particle capture surface and on a second particle capture surface disposed on said lid, wherein said second particle capture surface comprises a particle capture surface according to any one of claims 1-37 or a particle capture surface according to any one of claims 39-92.

115. The particle capture chamber of claim 114, wherein said first particle capture surface and said second particle capture surface are the same materials and configuration.

116. The particle capture chamber of claim 114, wherein said first particle capture surface and said second particle capture surface are the different materials and/or configuration.

117. The particle capture chamber according to any one of claims 114-116, wherein said lid is configured so that when closed, microchannels in said first particle capture surface are sealed, and when present in said second particle capture surface microchannels in said second particle capture surface are sealed.

118. The particle capture chamber according to any one of claims 114-117, wherein said sample chamber is configured to direct the flow of extractant fluid through the chamber so that the chemical/biochemical contents are dissolved in an extractant fluid volume smaller than the total volume of the chamber of said chamber without the microchannels present thereby concentrating said chemical/biochemical contents.

119. The particle capture chamber of claim 118, wherein the extractant fluid volume is less than 10%, or less than about 5%, or less than about 2% of the total volume of the chamber without the microchannels present.

120. The particle capture chamber according to any one of claims 118-119, wherein the concentration of analyte in said extractant fluid is increased by at least at least 2-fold, or at least about 5-fold, or at least about 10-fold, or at least about 20-fold as compared to the concentration of said analyte present in a volume of extractant fluid equal to the total volume of said chamber.

121. The particle capture chamber according to any one of claims 118-119, wherein the wherein the increase in concentration of the analyte provides for a 10-fold, or at least about a 20-fold improvement so that the extractant volume is 1/10 or 1/20 or less of the nominal volume of the chamber without the channels.

122. The particle capture chamber according to any one of claims of claim 118-121, wherein said first capture surface and, when present, said second capture surface, comprises channels that can be effectively washed with a total volume of extractant fluid of less than about 100 μL, or less than about 75 μL, or less than about 50 μL, or less than about 40 μL, or less than about 30 μL, or less than about 20 μL, or less than about 15 μL.

123. The particle capture chamber of claim 122, wherein said first capture surface and, when present, said second capture surface, comprises channels that can be effectively washed with a total volume of extractant fluid of as low as 10 μL or less.

124. The particle capture chamber according to any one of claims 114-123, wherein said sample chamber is configured with an inlet and outlet port and is configured to wash said first capture surface and, when present, said second capture surface, and deliver aerosol, and/or dust and/or ice particles, or components thereof to a chemical analysis system operably coupled to said sample chamber.

125. The particle capture system of claim 124, wherein said chamber is configured to deliver dust or ice particles to said chemical analysis system.

126. The particle capture chamber according to any one of claims 124-125, wherein said analysis system provides one or more analytic methods selected from the group consisting of optical microscopy, by optical spectroscopy, SEM, Raman spectroscopy, and mass spectrometry.

127. The particle capture chamber according to any one of claims 124-126, wherein said chemical analysis system comprise a programmable microfluidic analyzer (PMA) operably coupled to said capture chamber.

128. The particle capture chamber of claim 127, wherein said PMA comprises:
a plurality of pneumatic inputs;
a plurality of microfluidic channels; and
a plurality of μCE separation channels, where said pneumatic inputs microfluidic channels and μCE separation channels are configured to so that fluid samples enter and leave the processor through access ports at the bottom; wherein
an array of valves drive fluid routing on the PMA; and
sample and reagent storage is provided in addressable wells at the top.

129. The particle capture chamber of claim 128, wherein said PMA permits analysis of different samples.

130. The particle capture chamber according to any one of claims 109-129, wherein said capture surface comprises a component in an aircraft, a rocket, a satellite or space probe.

131. A method of detecting organic compounds in high velocity dust, aerosol, and/or ice particles, said method comprising:
providing a particle capture chamber according to any one of claims 97-107 in a high velocity particle plume where the lid of said particle capture chamber is open permitting particles comprising said plume to impact said first particle capture surface and, when present, said second particle capture surface to provide one or more surfaces with captured particles;
closing the lid of said particle capture chamber to define a closed sample chamber; and
analyzing said captured particles to identify presence and composition of organic molecules associated with said captured particles.

132. The method of claim 131, wherein said lid is open for at least a period of time sufficient to capture a detectable quantity of particles.

133. The method according to any one of claims 131-132, wherein said high velocity particle plume comprises an extraterrestrial particle plume.

134. The method of claim 133, wherein said high velocity particle plume comprises a particle plume at Europa or Enceladus.

135. The method according to any one of claims 131-132, wherein said particles comprise particles in a Venus cloud.

136. The method according to any one of claims 131-132, wherein said particles comprise comet debris.

137. The method according to any one of claims 131-132, wherein said particles comprise particles at high altitude.

138. The method according to any one of claims 131-132, wherein said particles comprise particles in low earth orbit.

139. The method according to any one of claims 131-138, wherein said analyzing comprises in situ analysis of said captured particles on said one or more capture surface(s).

140. The method of claim 139, wherein said in situ analysis comprises a spectroscopic analysis.

141. The method according to any one of claims 139-140, wherein said in situ analysis by one or more methods selected from the group consisting of SEM scanning, optical microscopy to identify absorption or emission of inorganic or organic materials, or detection of absorbance, fluorescence, phosphorescence or light scattering, or mass spectroscopy.

142. The method according to any one of claims 139-141, wherein said in situ analysis comprises Raman spectroscopy.

143. The method according to any one of claims 131-142, wherein said method comprises:
warming said sample chamber if necessary;
filling said sample chamber with a solvent or solvent system to suspend or dissolve organic molecules present on or in said particles;
transporting the suspended or dissolved organic molecules into a microfluidic processor; and
performing electrophoresis of said suspended or dissolved organic molecules in said microfluidic processor.

144. The method of claim 143, wherein said solvent or solvent system comprises water or a buffer.

145. The method of claim 143, wherein said solvent or solvent system comprises an aqueous two-phase partitioning system that partitions the analyte(s) (e.g., aerosol, and/or ice, and/or dust particles) or components thereof into one phase or into an interface between two phases comprising said partitioning system.

146. The method of claim 145, wherein said aqueous two-phase partitioning system comprises a system selected from the group consisting of oil/water systems, polymer/polymer systems, and polymer/salt systems.

147. The method according to any one of claims 145-146, wherein said two phase partitioning system comprises component 1 and component 2 as in Table 1.

148. The method of claim 147, wherein said two phase partitioning system comprises and oil/water system.

149. The method according to any one of claims 143-148, wherein said method comprises labeling one or more of amines, amino acids, carboxylic acids, aldehydes, ketones, and thiols with a fluorescent label.

150. The method according to any one of claims 143-149, wherein said electrophoresis comprises high-resolution capillary electrophoresis.

151. The method according to any one of claims 143-150, wherein said capillary electrophoresis comprises laser-induced fluorescence to detect the electrophoresed analytes.

152. The method according to any one of claims 143-151, wherein said capillary electrophoresis is performed by using a programmable microfluidic analyzer (PMA) operably coupled to said capture chamber.

153. The method of claim 152, wherein said PMA comprises:
a plurality of pneumatic inputs;
a plurality of microfluidic channels; and
a plurality of μCE separation channels, where said pneumatic inputs microfluidic channels and μCE separation channels are configured so that fluid samples enter and leave the processor through access ports at the bottom; wherein
an array of valves drive fluid routing on the PMA; and
sample and reagent storage is provided in addressable wells at the top.

154. The method of claim 153, wherein said PMA permits analysis of different samples.

155. The method according to any one of claims 131-154, wherein said method provides data about any proteinogenic, biotic and abiotic amino acid that informs decisions about possible life.

156. The method according to any one of claims 131-155, wherein said method detects, identifies and quantifies one or more of Ala, Asp, Glu, Gly, His, Leu, Ser, Val, beta-Ala, GABA, Iva, and AIB.

157. The method of claim 156, wherein said method detects, identifies and quantifies Ala, Asp, Glu, Gly, His, Leu, Ser, Val, beta-Ala, GABA, Iva, and AIB.

158. The method according to any one of claims 131-157, wherein said method provides at least 2% quantitation relative to glycine with a sensitivity of 2 femtomoles of captured organic target material (10 nM in 180 micrograms of captured ice).

159. The method according to any one of claims 131-158, wherein said method provides chiral amino acid separations by running a test set consisting of histidine, alanine, serine and Asp and or Glu along with one abiotic amino acid such as Iva.

160. A method of detecting organic compounds in high velocity dust, aerosol, and/or ice particles, said method comprising:
providing a particle capture chamber according to any one of claims 109-129 in a high velocity particle plume where the lid of said particle capture chamber is open permitting particles comprising said plume to impact said first particle capture surface and, when present, said second particle capture surface to provide one or more surfaces with captured particles;
closing the lid of said particle capture chamber to define a closed sample chamber where closing said lid creates a reduced volume sample chamber defined by features on said first particle capture surface, and when present said second particle capture surface; and
analyzing said captured particles to identify presence and composition of organic molecules associated with said captured particles.

161. The method of claim 160, wherein said lid is open for at least a period of time sufficient to capture a detectable quantity of particles.

162. The method according to any one of claims 160-161, wherein said high velocity particle plume comprises an extraterrestrial particle plume.

163. The method of claim 162, wherein said high velocity particle plume comprises a particle plume at Europa or Enceladus.

164. The method according to any one of claims 160-161, wherein said particles comprise particles in a Venus cloud.

165. The method according to any one of claims 160-161, wherein said particles comprise comet debris.

166. The method according to any one of claims 160-161, wherein said particles comprise particles at high altitude.

167. The method according to any one of claims 160-161, wherein said particles comprise particles in low earth orbit.

168. The method according to any one of claims 160-167, wherein said analyzing comprises in situ analysis of said captured particles on said one or more capture surface(s).

169. The method of claim 168, wherein said in situ analysis comprises mass spectroscopic analysis (e.g., laser adsorption mass spectrometry).

170. The method of claim 168, wherein said in situ analysis comprises a spectroscopic analysis.

171. The method according to any one of claims 168-170, wherein said in situ analysis one or more methods selected from the group consisting of SEM scanning, optical microscopy to identify absorption, light scattering or emission of inorganic or organic materials, or detection of fluorescence or phosphorescence.

172. The method according to any one of claims 168-171, wherein said in situ analysis comprises Raman spectroscopy.

173. The method according to any one of claims 160-172, wherein said method comprises:
warming said sample chamber if necessary;
filling said sample chamber with a solvent or solvent system to suspend or dissolve organic molecules present on or in said particles;
transporting the suspended or dissolved organic molecules into a microfluidic processor; and
performing electrophoresis of said suspended or dissolved organic molecules in said microfluidic processor.

174. The method of claim 173, wherein said solvent or solvent system comprises water or a buffer.

175. The method of claim 173, wherein said solvent or solvent system comprises an aqueous two-phase partitioning system that partitions the analyte(s) (e.g., aerosol, and/or ice, and/or dust particles) or components thereof into one phase or into an interface between two phases comprising said partitioning system.

176. The method of claim 175, wherein said aqueous two-phase partitioning system comprises a system selected from the group consisting of oil/water systems, polymer/polymer systems, and polymer/salt systems.

177. The method according to any one of claims 175-176, wherein said two phase partitioning system comprises component 1 and component 2 as in Table 1.

178. The method of claim 177, wherein said two phase partitioning system comprises and oil/water system.

179. The method according to any one of claims 173-178, wherein said filling said sample chamber with a solvent or solvent system comprises washing said one or more channels with a volume of less than about 100 μL, or less than about 75 μL, or less than about 50 μL, or less than about 40 μL, or less than about 30 μL, or less than about 20 μL, or less than about 15 μL of said solvent or solvent system.

180. The particle capture surface of claim 179, wherein said filling said sample chamber with a solvent or solvent system comprises washing said one or more channels with a volume as small as 10 μL or less.

181. The method according to any one of claims 179-180, wherein said volume is fluid volume smaller than the total volume of the chamber of said sample chamber without the microchannels present thereby concentrating said chemical/biochemical contents.

182. The particle capture chamber of claim 181, wherein the volume is less than 10%, or less than about 5%, or less than about 2% of the total volume of the chamber of said chamber without the microchannels present.

183. The particle capture chamber according to any one of claims 179-182, wherein the concentration of analyte in said extractant fluid is increased by at least at least 2-fold, or at least about 5-fold, or at least about 10-fold, or at least about 20-fold as compared to the concentration of said analyte present in a volume of extractant fluid equal to the total volume of said chamber.

184. The method according to any one of claims 173-180, wherein said method comprises labeling one or more of amines, amino acids, carboxylic acids, aldehydes, ketones, thiols, and polycyclic aromatic hydrocarbons (PAHs) with a fluorescent label.

185. The method according to any one of claims 173-184, wherein said electrophoresis comprises high-resolution capillary electrophoresis.

186. The method according to any one of claims 173-185, wherein said capillary electrophoresis comprises laser-induced fluorescence to detect the electrophoresed analytes.

187. The method according to any one of claims 173-186, wherein said capillary electrophoresis is performed by a programmable microfluidic analyzer (PMA) operably coupled to said capture chamber.

188. The method of claim 187, wherein said PMA comprises:
a plurality of pneumatic inputs;
a plurality of microfluidic channels; and
a plurality of μCE separation channels, where said pneumatic inputs microfluidic channels and μCE separation channels are configured to so that fluid samples enter and leave the processor through access ports at the bottom; wherein
an array of valves drive fluid routing on the PMA; and
sample and reagent storage is provided in addressable wells at the top.

189. The method of claim 188, wherein said PMA permits analysis of different samples.

190. The method according to any one of claims 160-189, wherein said method provides data about any proteinogenic, biotic and abiotic amino acid that informs decisions about possible life.

191. The method according to any one of claims 160-190, wherein said method detects, identifies and quantifies one or more of Ala, Asp, Glu, Gly, His, Leu, Ser, Val, beta-Ala, GABA, Iva, and AIB.

192. The method of claim 191, wherein said method detects, identifies and quantifies Ala, Asp, Glu, Gly, His, Leu, Ser, Val, beta-Ala, GABA, Iva, and AIB.

193. The method according to any one of claims 160-192, wherein said method provides at least 2% quantitation relative to glycine with a sensitivity of 2 femtomoles of captured organic target material (10 nM in 180 micrograms of captured ice).

194. The method according to any one of claims 160-193, wherein said method provides chiral amino acid separations by running a test set consisting of histidine, alanine, serine and Asp and or Glu along with one abiotic amino acid such as Iva.
